SEQUENCE LISTING (15 sequences)

SEQ ID NO: 1 - protein, 5 aa, synthetic construct
RPQGF

SEQ ID NO: 2 - protein, 8 aa, synthetic construct
RPQGFGPP

SEQ ID NO: 3 - protein, 123 aa, synthetic construct
QVQLLESGGGVVQPGGSLRLSCAASGFAFSIYDMNWVRQAPGKGLEWVSAISSGGGTTYYADSVKGRFTISRDNAKNSLYLQMNSLRAEDTAVYYCARHSGYGTHWGVLFAYWGRGTLVTVSS

SEQ ID NO: 4 - protein, 107 aa, synthetic construct
DIQMTQSPSSLSASVGDRVTITCRASQDIHGYLNWYQQKPGKAPKLLIYYTSILHSGVPSRFSGSGSGTDFTLTISSLQPEDFATYFCQQGSTLPWTFGQGTKLEIK

SEQ ID NO: 5 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 8 aa, synthetic construct
GFAFSIYD

SEQ ID NO: 7 - protein, 8 aa, synthetic construct
ISSGGGTT

SEQ ID NO: 8 - protein, 16 aa, synthetic construct
ARHSGYGTHWGVLFAY

SEQ ID NO: 9 - protein, 6 aa, synthetic construct
QDIHGY

SEQ ID NO: 10 - skipped

SEQ ID NO: 11 - protein, 9 aa, synthetic construct
QQGSTLPWT

SEQ ID NO: 12 - protein, 5 aa, synthetic construct
LLQGG

SEQ ID NO: 13 - protein, 8 aa, synthetic construct
GGGLLQGG

SEQ ID NO: 14 - protein, 460 aa, synthetic construct
QVQLLESGGGVVQPGGSLRLSCAASGFAFSIYDMNWVRQAPGKGLEWVSAISSGGGTTYYADSVKGRFTISRDNAKNSLYLQMNSLRAEDTAVYYCARHSGYGTHWGVLFAYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRPQGFGPP

SEQ ID NO: 15 - protein, 214 aa, synthetic construct
DIQMTQSPSSLSASVGDRVTITCRASQDIHGYLNWYQQKPGKAPKLLIYYTSILHSGVPSRFSGSGSGTDFTLTISSLQPEDFATYFCQQGSTLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0216358 A1
(43) Pub. Date: Jul. 30, 2026

(54) METHODS OF TREATING CANCER USING ANTI-CD22 ANTIBODY OLIGONUCLEOTIDE CONJUGATES

(71) Applicant: Tallac Therapeutics, Inc., Burlingame, CA (US)

(72) Inventors: Hong I. WAN, Foster City, CA (US); Ons HARRABI, Burlingame, CA (US); Kevin Nelson HELLER, Potomac, MD (US)

(21) Appl. No.: 19/645,017

(22) Filed: Apr. 10, 2026

Related U.S. Application Data
(63) Continuation of application No. PCT/US2024/051054, filed on Oct. 11, 2024.
(60) Provisional application No. 63/590,302, filed on Oct. 13, 2023.

(51) Int. Cl. A61K 47/68; A61K 47/02; A61K 47/12; A61K 47/18; A61K 47/26; A61P 35/00

(52) U.S. Cl. CPC A61K 47/6807; A61K 47/02; A61K 47/12; A61K 47/183; A61K 47/26; A61K 47/6849; A61K 47/6889; A61P 35/00

(57) ABSTRACT

The present disclosure provides for methods for treating cancer using anti-CD22 antibody-oligonucleotide conjugates.

### CROSS-REFERENCE TO RELATED APPLICATIONS

This application is a continuation of International Application No. PCT/US2024/051054, filed Oct. 11, 2024, which claims the priority benefit of U.S. Provisional Application No. 63/590,302, filed Oct. 13, 2023, which is hereby incorporated by reference in its entirety.

### REFERENCE TO AN ELECTRONIC SEQUENCE LISTING

The content of the electronic sequence listing (396152000801SeqList.xml; Size: 14,533 bytes; and Date of Creation: Apr. 7, 2026) is incorporated herein by reference in its entirety.

### FIELD OF INVENTION

The present disclosure relates generally to methods for treating cancer using anti-CD22 antibody-oligonucleotide conjugates.

### BACKGROUND

Immune-mediated elimination of cancer requires the coordination of the innate (e.g. dendritic cells, macrophages, myeloid derived suppressor cells) and adaptive (i.e. B cells, cytotoxic T cells, regulatory T cells) immune systems. Current standard of care cancer treatments include immunotherapeutics modulating the immune system to fight cancer. The basis of most immunotherapy treatments involve the administration of immune checkpoint blockade therapies, predominantly the anti-PD-1, anti-CTLA-4 antibodies. However, these therapies are not effective in all patients. As well, such therapies predominantly act specifically to increase T lymphocyte activity, thereby neglecting the importance of coordinated immune response. Thus, there remains a need for a safe and effective treatment that utilizes the broad scope of possible immune involvement in cancer elimination.

### BRIEF SUMMARY

In one aspect, provided herein is a method of treating cancer in an individual comprising administering to the individual a conjugate comprising a compound of Formula I (also referred to herein as “Compound 1”) at a dose of between 3 mg/kg to 30 mg/kg, where the compound of Formula I comprises an anti-CD22 antibody (Ab) and an immunomodulating oligonucleotide (P) wherein the Ab comprises two antibody light chains, two antibody heavy chains, and two Q-tag peptides (Q); wherein each of the two Q-tag peptides comprises the amino acid sequence RPQGFGPP (SEQ ID NO:2); wherein one Q-tag peptide is linked to the C-terminus of each of the two antibody heavy chains; wherein one of the two Q-tag peptides is linked to the immunomodulating oligonucleotide via an amide bond with the glutamine residue of the Q-tag peptide and a linker (L) as shown in Formula (A):

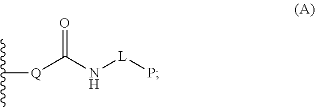

wherein [symbol] indicates the point of attachment of Q to the antibody (Ab); wherein each heavy chain of the Ab comprises a CDR1 comprising the amino acid sequence of GFAFSIYD (SEQ ID NO:6), a CDR2 comprising the amino acid sequence of ISSGGGTT (SEQ ID NO:7), and a CDR3 comprising the amino acid sequence of ARHSGYGTHWGVLFAY (SEQ ID NO:8), and each light chain of the Ab comprises a CDR1 comprising the amino acid sequence of QDIHGY (SEQ ID NO:9), a CDR2 comprising the amino acid sequence of YTS, and a CDR3 comprising QQGSTLPWT (SEQ ID NO:11); wherein the linker L is

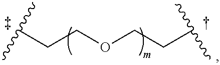

wherein m is 24, and wherein [symbol] indicates the point of attachment to the oligonucleotide P, and [symbol] indicates the point of attachment to the glutamine residue of the Q-tag; and wherein the oligonucleotide P comprises the structure:

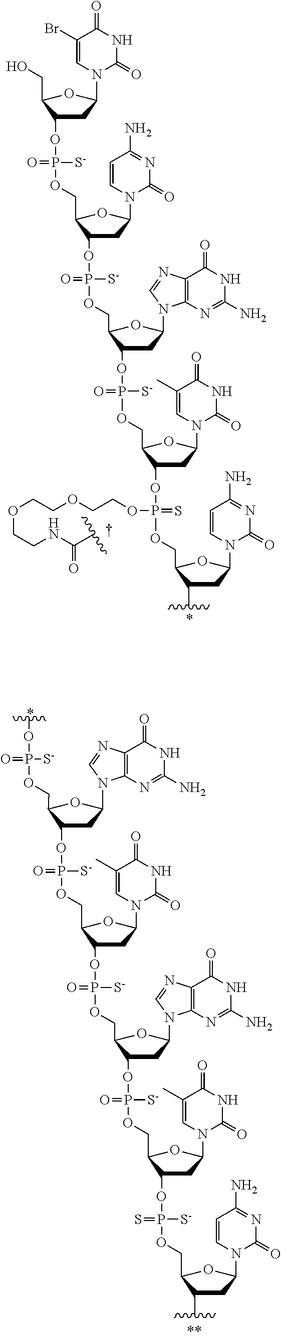

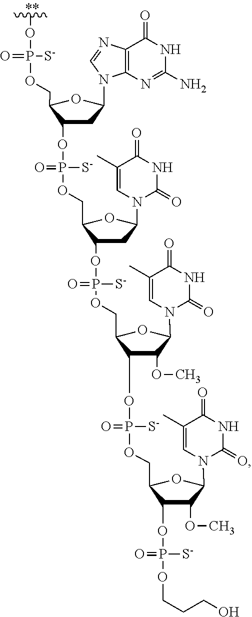

or a pharmaceutically acceptable salt thereof,
wherein [symbol] and [symbol] ** indicate the points of attachment within the oligonucleotide, wherein [symbol] indicates the point of attachment to the linker L.

In some embodiments, the method further comprises administering an anti-PD-1 antibody to the individual. In some embodiments, the anti-PD-1 antibody is pembrolizumab. In some embodiments, the pembrolizumab is administered to the individual once every three weeks. In some embodiments, the pembrolizumab is administered to the individual at a dose of 200 mg. In some embodiments, the method further comprises administering an anti-PD-1 antibody to the individual, wherein the anti-PD-1 antibody is pembrolizumab, and wherein the pembrolizumab is administered to the individual at a dose of 200 mg once every three weeks and the compound of Formula I is administered to the individual once every three weeks at a dose of 0.3 mg/kg, 1.0 mg/kg, 3.0 mg/kg, or 6.0 mg/kg.

In some embodiments, the individual to which the compound of Formula I is administered has a solid tumor that is inoperable, locally advanced, metastatic, and/or recurrent. In some embodiments, the individual has a cancer selected from the group consisting of melanoma, cutaneous melanoma, breast cancer, ovarian cancer, colorectal cancer, non-small cell lung cancer (NSCLC), renal cell carcinoma (RCC), head and neck squamous cell carcinoma (HNSCC), Merkel cell carcinoma, hepatocellular carcinoma (HCC), cervical squamous cell carcinoma, urothelial carcinoma, endometrial carcinoma, gastro-esophageal adenocarcinoma, and cholangiocarcinoma. In some embodiments, the individual is diagnosed or has cutaneous melanoma. In some embodiments, tumor cells of the cutaneous melanoma comprise a genetic mutation. In some embodiments, the genetic mutation is a mutation in one or more of BRAF, NTRK and KIT. In some embodiments, tumor cells of the cutaneous melanoma do not comprise a genetic mutation. In some embodiments, the individual is diagnosed or has a biliary tract cancer. In some embodiments, the biliary tract cancer is intra- or extrahepatic cholangiocarcinoma. In some embodiments, the biliary tract cancer is a gall bladder cancer.

In some embodiments, the individual to which the compound of Formula I is administered has an immunogenic solid tumor. In some embodiments, the immunogenic solid tumor is a gastric cancer, a cancer of the stomach and gastroesophageal junction, a non-small cell lung cancer (NSCLC), renal cell carcinoma (RCC), urothelial cancer, cutaneous squamous cell carcinoma, Merkel cell carcinoma, or a tumor with one or more of known microsatellite instable (MSI-H), mismatch repair (MMMR) deficient, and tumor mutation burden high.

In some embodiments, the individual to which the compound of Formula I is administered has a solid tumor or tumor cells with one or more genetic mutations. In some embodiments, the genetic mutation is a mutation in one or more of HER2, BRAF, FGFR, IDH1, NTRK, RET, KIT, ROS1, NRAS, ALK, MET, and KRAS.

In some embodiments, the individual to which the compound of Formula I is administered has a solid tumor that has progressed on and/or is intolerant to standard therapy. In some embodiments, the cancer has progressed, or the cancer has recurred after prior treatment with an immune-oncology agent. In some embodiments, the immune-oncology agent is an anti-PD-1 antibody or an anti-CTLA4 antibody.

In some embodiments, the compound of Formula I is administered to the individual at a dose of between 3 mg/kg to 30 mg/kg. In some embodiments, the compound of Formula I is administered to the individual at a dose of about 3.0, 6.0, 12.0, 18.0, 24.0, or 30.0 mg/kg. In some embodiments, the compound of Formula I is administered to the individual at a dose of about 3.0, 6.0, 12.0, 18.0, 24.0, or 30.0 mg/kg once every two weeks. In some embodiments, the compound of Formula I is administered to the individual at a dose of about 3.0, 6.0, 12.0, 18.0, 24.0, or 30.0 mg/kg once every four weeks. In some embodiments, the compound of Formula I is administered to the individual at a dose of about 3.0, 6.0, 12.0, 18.0, 24.0, or 30.0 mg/kg once every three weeks, such as when combined with a second therapy having a dosing schedule of once every three weeks.

In some embodiments, the compound of Formula I is administered to the individual at a dose of about 3.0, 6.0, 12.0, 18.0, 24.0, or 30.0 mg/kg and each heavy chain of the Ab of the compound of Formula comprises a heavy chain variable region comprising the amino acid sequence of SEQ ID NO:3. In some embodiments, the compound of Formula I is administered to the individual at a dose of about 3.0, 6.0, 12.0, 18.0, 24.0, or 30.0 mg/kg and each light chain of the Ab of the compound of Formula I comprises a light chain variable region comprising the amino acid sequence of SEQ ID NO:4. In some embodiments, each heavy chain of the Ab including the Q-tag peptide comprises the amino acid sequence of SEQ ID NO: 14; and wherein each light chain of the Ab comprises the amino acid sequence of SEQ ID NO: 15. In some embodiments, the individual is a human. In some embodiments, the compound of Formula I is administered in a formulation comprising the conjugate at a concentration of 30 mg/mL; 20 mM citrate; 150 mM L-arginine; 50 mM NaCl; and 0.02% polysorbate 80 (w/v); wherein the formulation is at pH 6.5. In some embodiments, the compound of Formula I is administered to the individual at a dose of about 3.0, 6.0, 12.0, 18.0, 24.0, or 30.0 mg/kg and administered to the individual intravenously.

### DESCRIPTION OF THE FIGURES

The present application can be understood by reference to the following description taken in conjunction with the accompanying figures.

FIG. 1 shows a graph of CD22 internalization by measuring CD22 expression at the cell surface from patient samples in dosing cohorts administered the compound of Formula I at 0.1, 0.3, 1, and 3 mg/kg.

FIG. 2A shows CD86 expression on naïve B cells after treatment with the compound of Formula I in patients at the indicated doses.

FIG. 2B shows CD86 expression on memory B cells after treatment with the compound of Formula I in patients at the indicated doses.

FIG. 2C shows CD40 expression on naïve B cells after treatment with the compound of Formula I in patients at the indicated doses.

FIG. 2D shows CD40 expression on memory B cells after treatment with the compound of Formula I in patients at the indicated doses.

FIG. 3A and FIG. 3B show the correlation between CD22 internalization and CD86 expression on naïve B cells in patients dosed at 0.3 mg/kg (FIG. 3A) and 3 mg/kg (FIG. 3B).

FIGS. 4A-4C show the levels of cytokines and chemokines in serum of patients dosed at the levels indicated.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Provided herein are methods of treating solid tumors comprising administering a conjugate comprising the compound of Formula I to an individual at dose between 3.0 mg/kg to 30 mg/kg.

Toll-like receptor 9 (TLR9), also designated as CD289, is a transmembrane and intracellular pattern recognition receptor that recognizes pathogen-associated molecular patterns (PAMPs) to engage an immune response in the absence of pathogens. TLR9 is an important receptor expressed by immune cells including dendritic cells (DCs), B lymphocytes, macrophages, natural killer cells, and other antigen presenting cells. TLR9 activation triggers intracellular signaling cascades that lead to activation, maturation, proliferation, and cytokine production in these immune cells, thus bridging and coordinating innate and adaptive immunity. Martinez-Campos et al., Viral Immunol. 2016, 30, 98-105; Notley et al., Sci. Rep. 2017, 7, 42204. Thus, the identification of novel TLR9 agonist compounds may activate both innate and adaptive immunity and serve as potential anti-cancer therapeutics.

Natural TLR9 agonists include unmethylated cytosine-guanine dinucleotide (CpG)-containing oligodeoxynucleotides (CpG ODNs). CpG ODNs are often susceptible to degradation in serum and thus pharmacokinetics of CpG ODNs may be one of the limiting factors in their development as therapeutics. Additionally, CpG ODNs exhibit uneven tissue distribution that can result in increased PAMP-associated toxicity. These shortcoming(s) can be overcome by conjugating the CpG ODN to a targeting moiety for the treatment of a specific disease, e.g. conjugation to an anti-CD22 antibody for anti-cancer therapy. Indeed, CD22 is a transmembrane sialoglycoprotein receptor with restricted expression on B cells, including tumor-infiltrating B cells (TIBs) that will internalize in complex with an anti-CD22-CpG conjugate, thereby delivering TLR9 agonist to stimulate an anti-cancer immune response. Preclinical murine tumor models showed that an anti-CD22-CpG conjugate activated the TLR9 pathway in B cells to increase immune activation, cytokine production, antigen presentation, and anti-tumor efficacy. However, there remains a need to assess the safety and efficacy of this drug compound in human cancer patients.

### I. Definitions

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Throughout this application, unless the context indicates otherwise, references to a compound of Formula I include ionic forms, polymorphs, pseudopolymorphs, amorphous forms, solvates, co-crystals, chelates, isomers, tautomers, oxides (e.g., N-oxides, S-oxides), esters, prodrugs, isotopes and/or protected forms thereof. In some embodiments, references to a compound of Formula I include polymorphs, solvates, co-crystals, isomers, tautomers and/or oxides thereof.

The term “immunomodulating polynucleotide” or “immunomodulating oligonucleotide” as used herein, represents a polynucleotide construct containing a total of from 6 to 50 contiguous nucleosides. The immunomodulating polynucleotides are capable of modulating an innate immune response, as determined by, e.g., a change in the activation of intracellular signaling pathway(s) including but not limited to NFκB, a change in the expression of an activation marker or a change in the secretion of at least one inflammatory cytokine or at least one type I interferon in an immune cell (e.g., antigen-presenting cell) to which an immunomodulating polynucleotide was delivered (e.g., in comparison to another immune cell (e.g., antigen-presenting cell) to which an immunomodulating polynucleotide was not delivered) or in an immune cell that interacts with an immune cell (e.g., antigen-presenting cell) to which an immunomodulating polynucleotide was delivered (including direct cell-to-cell interactions as well as indirect stimulation, e.g., from one or more cytokines secreted by the cell to which an immunomodulating polynucleotide was delivered).

It is to be understood that the terms “polynucleotide” and “oligonucleotide” may be used interchangeably herein. It is further to be understood that the terms “immunomodulating polynucleotide,” and “conjugate” encompass salts of the immunomodulating polynucleotide and conjugate, respectively. For example, the terms “immunomodulating polynucleotide,” and “conjugate” encompasses both the protonated, neutral form (P-XU moiety, where X is O or S) of a phosphate, phosphorothioate, or phosphorodithioate and the deprotonated, ionic form (P-X-moiety, where X is O or S) of a phosphate, phosphorothioate, or phosphorodithioate.

As used herein, the term “amino acid” refers to any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, omithine, hypusine, 2-aminoisobutyric acid, dehydroalanine, gamma-aminobutyric acid, citrulline, beta-alanine, alpha-ethyl-glycine, alpha-propyl-glycine and norleucine.

The terms “antibody,” “immunoglobulin,” and “Ig” are used interchangeably herein, and are used in the broadest sense and specifically cover, for example, individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies. An antibody can be human, humanized, chimeric and/or affinity matured as well as an antibody from other species, for example, mouse and rabbit.

The term “antibody” is intended to include a polypeptide product of B cells within the immunoglobulin class of polypeptides that is able to bind to a specific antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxyl-terminal portion of each chain includes a constant region. See Borrebaeck (ed.) (1995) Antibody Engineering, Second Ed., Oxford University Press.; Kuby (1997) Immunology, Third Ed., W. H. Freeman and Company, New York. Antibodies also include, but are not limited to, synthetic antibodies, monoclonal antibodies, recombinant antibodies, multispecific antibodies (including bi-specific antibodies), human antibodies, humanized antibodies, camelized antibodies, chimeric antibodies, intrabodies, anti-idiotypic (anti-Id) antibodies, and functional fragments thereof, which refers a portion of an antibody heavy or light chain polypeptide that retains some or all of the binding activity of the antibody from which the fragment is derived. Non-limiting examples of functional fragments of an antibody include single-chain Fvs (scFv) (e.g., including monospecific or bispecific), Fab fragments, F(ab′) fragments, F(ab)₂ fragments, F(ab′)₂ fragments, disulfide-linked Fvs (sdFv), Fd fragments, Fv fragments, scRv-Fc, nanobody, diabody, triabody, tetrabody, and minibody. In some embodiments, the antibody comprises an Fc variant that has reduced or ablated effector function. In particular, antibodies provided herein include immunoglobulin molecules and immunologically active portions of immunoglobulin molecules, for example, antigen-binding domains or molecules that contain an antigen-binding site that binds to the antigen (e.g., one or more complementarity determining regions (CDRs) of an anti-CD56 antibody or an anti-SIRPα antibody). Such antibody fragments are described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics 1993, 22, 189-224; Plückthun and Skerra, Meth. Enzymol. 1989, 178, 497-515; and Day, Advanced Immunochemistry, Second Ed., Wiley-Liss, Inc., New York, NY (1990). The antibodies provided herein can be of any type (e.g., IgG, IgE, IgM, IgD, IgA, and IgY), any class (e.g., IgG1, IgG2, IgG3, IgG4, IgAQ1, and IgA2), or any subclass (e.g., IgG2a and IgG2b) of an immunoglobulin molecule.

The term “antigen” refers to a predetermined target to which an antibody can selectively bind. A target antigen can be a polypeptide, carbohydrate, nucleic acid, lipid, hapten, or fragment thereof, or other naturally occurring or synthetic compound. In one embodiment, the target antigen is a polypeptide.

The terms “antigen-binding fragment,” “antigen-binding domain,” and “antigen-binding region” refer to a portion of an antibody that comprises the amino acid residues that interact with an antigen (e.g., a polypeptide, carbohydrate, nucleic acid, lipid, hapten, or fragment thereof, or other naturally occurring or synthetic compound) and confer on the binding agent its specificity and affinity for the antigen (e.g., complementarity determining regions (CDRs)).

The term “specific binding,” “specifically binds to,” or “specific for” a particular polypeptide or an epitope on a particular polypeptide target can be exhibited, for example, by a molecule (e.g., an antibody) having a dissociation constant (K_{d}) for the target of at least about 10⁻⁴ M, at least about 10⁻⁵ M, at least about 10⁻⁶ M, at least about 10⁻⁷ M, at least about 10⁻⁸ M, at least about 10⁻⁹ M, at least about 10⁻¹⁰ M, at least about 10⁻¹¹ M, or at least about 10⁻¹² M. In one embodiment, the term “specific binding” refers to binding where a molecule binds to a particular polypeptide or epitope on a particular polypeptide without substantially binding to any other polypeptide or polypeptide epitope.

A 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, see, e.g., Basic and Clinical Immunology, 8th edition, Stites et al. (eds.), Appleton & Lange, Norwalk, CT, 1994, page 71 and Chapter 6.

The term “variable region” or “variable domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a R sheet configuration, connected by three hypervariable regions, which form loops connecting, and in some cases forming part of, the R sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “variable region residue numbering as in Kabat” or “amino acid position numbering as in Kabat”, and variations thereof, refers to the numbering system used for heavy chain variable regions or light chain variable regions of the compilation of antibodies in Kabat et al., Sequences of Proteins of Immunological Interest, 5th Ed. Public Health Service, National Institutes of Health, Bethesda, MD. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, including, for example, by AbM, Chothia, Contact, IMGT and AHon.

An “intact” antibody is one comprising an antigen-binding site as well as a CL and at least heavy chain constant regions, CH1, CH2 and CH3. The constant regions may include human constant regions or amino acid sequence variants thereof. Preferably, an intact antibody has one or more effector functions.

The term “antibodyfragment” refers to a portion of an intact antibody, preferably the antigen-binding or variable region of the intact antibody. Examples of antibody fragments include, without limitation, Fab, Fab′, F(ab′)₂, and Fv fragments; diabodies and di-diabodies (see, e.g., Holliger et al., Proc. Natl. Acad. Sci. U.S.A. 1993, 90, 6444-8; Lu et al., J. Biol. Chem. 2005, 280, 19665-72; Hudson et al., Nat. Med. 2003, 9, 129-134; WO 93/11161; and U.S. Pat. Nos. 5,837,242 and 6,492,123); single-chain antibody molecules (see, e.g., U.S. Pat. Nos. 4,946,778; 5,260,203; 5,482,858 and 5,476,786); dual variable domain antibodies (see, e.g., U.S. Pat. No. 7,612,181); single variable domain antibodies (SdAbs) (see, e.g., Woolven et al., Immunogenetics 1999, 50, 98-101 Streltsov et al., Proc. Natl. Acad. Sci. U.S.A. 2004, 101, 12444-12449); and multispecific antibodies formed from antibody fragments.

The term “functional fragment,” “binding fragment,” or “antigen-binding fragment” of an antibody refers to a molecule that exhibits at least one of the biological functions attributed to the intact antibody, the function comprising at least binding to the target antigen.

The term “heavy chain” when used in reference to an antibody refers to a polypeptide chain of about 50-70 kDa, wherein the amino-terminal portion includes a variable region of about 120 to 130 or more amino acids and a carboxyl-terminal portion that includes a constant region. The constant region can be one of five distinct types, (e.g., isotypes) referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while p and F contain approximately 550 amino acids. When combined with a light chain, these distinct types of heavy chains give rise to five well known classes (e.g., isotypes) of antibodies, IgA, IgD, IgE, IgG and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxyl-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only a beta klotho epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., Nature 1975, 256, 495; or may be made using recombinant DNA methods in bacterial, eukaryotic animal or plant cells (see, e.g., U.S. Pat. No. 4,816,567). The “monoclonal antibodies” may also be isolated from phage antibody libraries using the techniques described in Clackson et al., Nature 1991, 352, 624-628 and Marks et al., J. Mol. Biol. 1991, 222, 581-597, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Exemplary methods of producing monoclonal antibodies are provided in the Examples herein.

“Humanized” forms of nonhuman (e.g., murine) antibodies are chimeric antibodies that include human immunoglobulins (e.g., recipient antibody) in which the native CDR residues are replaced by residues from the corresponding CDR of a nonhuman species (e.g., donor antibody) such as mouse, rat, rabbit or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 1986, 321, 522-525; Riechmann et al., Nature 1988, 332, 323-329; Presta, Curr. Opin. Biotechnol. 1992, 3, 394-398; Carter et al., Proc. Natl. Acad. Sci. U.S.A. 1992, 89, 4285-4289; and U.S. Pat. Nos. 6,800,738, 6,719,971, 6,639,055, 6,407,213, and 6,054,297.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 1991, 227, 381; Marks et al., J. Mol. Biol. 1991, 222, 581) and yeast display libraries (Chao et al., Nature Protocols 2006, 1, 755-768). Also available for the preparation of human monoclonal antibodies are methods described in Cole et al., Monoclonal Antibodies and Cancer Therapy, Alan R. Liss, p. 77 (1985); Boerner et al., J. Immunol. 1991, 147, 86-95. See also van Dijk and van de Winkel, Curr. Opin. Pharmacol. 2001, 5, 368-374. Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 1995, 6, 561-566; Bruggemann and Taussing, Curr. Opin. Biotechnol. 1997, 8, 455-458; and U.S. Pat. Nos. 6,075,181 and 6,150,584 regarding XENOMOUSE™ technology). See also, for example, Li et al., Proc. Natl. Acad. Sci. U.S.A. 2006, 103, 3557-3562 regarding human antibodies generated via a human B-cell hybridoma technology.

A “CDR” refers to one of three hypervariable regions (H1, H2, or H3) within the non-framework region of the immunoglobulin (Ig or antibody) VH β-sheet framework, or one of three hypervariable regions (L1, L2, or L3) within the non-framework region of the antibody VL j-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains. Kabat et al., J. Biol. Chem. 1977, 252, 6609-6616; Kabat, Adv. Protein Chem. 1978, 32, 1-75. CDR region sequences also have been defined structurally by Chothia as those residues that are not part of the conserved β-sheet framework, and thus are able to adapt different conformations. Chothia and Lesk, J. Mol. Biol. 1987, 196, 901-917. Both terminologies are well recognized in the art. CDR region sequences have also been defined by AbM, Contact and IMGT. The positions of CDRs within a canonical antibody variable region have been determined by comparison of numerous structures. Al-Lazikani et al., J. Mol. Biol. 1997, 273, 927-948; Morea et al., Methods. 2000, 20, 267-279. Because the number of residues within a hypervariable region varies in different antibodies, additional residues relative to the canonical positions are conventionally numbered with a, b, c and so forth next to the residue number in the canonical variable region numbering scheme. Al-Lazikani et al., supra (1997). Such nomenclature is similarly well known to those skilled in the art.

The term “hypervariable region”, “HVR”, or “H”, when used herein refers to the regions of an antibody variable region that are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (see, e.g., Kabat et al., Sequences of Proteins of Immunological Interest, 5th Ed. Public Health Service, National Institutes of Health, Bethesda, MD. (1991)). Chothia refers instead to the location of the structural loops. See, e.g., Chothia and Lesk, J. Mol. Biol. 1987, 196, 901-917. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

The term “DAR” refers to a drug-antibody ratio of an oligonucleotide-antibody conjugate, more specifically an immunomodulating polynucleotide-antibody ratio. In some instances, for example, an oligonucleotide-antibody conjugate may be described herein as having a DAR of 1 or as a DAR1 conjugate, wherein the oligonucleotide-antibody ratio is 1-to-1. In other instances, an oligonucleotide-antibody conjugate may be described herein as having a DAR of 2 or as a DAR2 conjugate, wherein the oligonucleotide-antibody ratio is 2-to-1.

As used herein, the terms “including,” “containing,” and “comprising” are used in their open, non-limiting sense. It is also understood that aspects and embodiments of the invention described herein may include “consisting” and/or “consisting essentially of” aspects and embodiments.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

As used herein, a “carrier” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™.

As used herein, the term “effective amount” or “therapeutically effective amount” of a substance is at least the minimum concentration required to bring about a measurable improvement (e.g., such as towards a therapeutic endpoint) or prevention of a particular disorder. An effective amount herein may vary according to factors such as the disease state, age, sex, and weight of the patient, and the ability of the substance to elicit a desired response in the individual. An effective amount is also one in which any toxic or detrimental effects of the treatment are outweighed by the therapeutically beneficial effects. In reference to cancer, an effective amount comprises an amount sufficient to cause a tumor to shrink and/or to decrease the growth rate of the tumor (such as to suppress tumor growth) or to prevent or delay other unwanted cell proliferation in cancer or to reach a particular threshold for a response criteria. In some embodiments, an effective amount is an amount sufficient to delay development of cancer. In some embodiments, an effective amount is an amount sufficient to prevent or delay recurrence. In some embodiments, an effective amount is an amount sufficient to reduce recurrence rate in the individual. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) reduce recurrence rate of tumor, and/or (viii) relieve to some extent one or more of the symptoms associated with the cancer. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

The terms “protein,” “polypeptide” and “peptide” are used herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Typically, a protein for use herein will have a molecular weight of at least about 5-20 kDa, alternatively at least about 20-100 kDa, or at least about 100 kDa. Also included within the definition are, for example, proteins containing one or more analogs of an amino acid (including, for example, unnatural amino acids, etc.), as well as other modifications known in the art.

A “pharmaceutically acceptable salt” is a salt form that is non-toxic, biologically tolerable, or otherwise biologically suitable for administration to the subject. See generally Berge et al. (1977) J. Pharm. Sci. 66, 1. Particular pharmaceutically acceptable salts are those that are pharmacologically effective and suitable for contact with the tissues of subjects without undue toxicity, irritation, or allergic response. Pharmaceutically acceptable salts include, without limitation, acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like. These salts may be derived from inorganic or organic acids. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, y-hydroxybutyrates, glycolates, tartrates, and mandelates. In some embodiments, pharmaceutically acceptable salts are formed when an acidic proton present in the parent compound either is replaced by a metal ion, e.g., an alkali metal ion, an alkaline earth ion, or an aluminum ion; or coordinates with an organic base. Salts derived from pharmaceutically acceptable organic non-toxic bases include, without limitation, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimetharnine, dicyclohexylamine, caffeine, procaine, hydrabamine, choline, betaine, ethylenediamine, glucosamine, N-ethylglucamine, N-methylglucamine, theobromine, purines, piperazine, piperidine, N-ethylpiperidine, polyamine resins, amino acids such as lysine, arginine, histidine, and the like. Examples of pharmaceutically acceptable base addition salts include those derived from inorganic bases such as sodium, potassium, lithium, ammonium, calcium, magnesium, iron, zinc, copper, manganese, aluminum salts and the like. In some embodiments, the organic non-toxic bases are L-amino acids, such as L-lysine and L-arginine, tromethamine, N-ethylglucamine and N-methylglucamine. Acceptable inorganic bases include, without limitation, aluminum hydroxide, calcium hydroxide, potassium hydroxide, sodium carbonate, sodium hydroxide, and the like. Lists of other suitable pharmaceutically acceptable salts are found in Remington's Pharmaceutical Sciences, 17th Edition, Mack Publishing Company, Easton, Pa., 1985.

A “solvate” is formed by the interaction of a solvent and a compound. Suitable solvents include, for example, water and alcohols (e.g., ethanol). Solvates include hydrates having any ratio of compound to water, such as monohydrates, dihydrates and hemi-hydrates.

A “subject,” “patient” or “individual” is a human. The individual can be male or female and can be any suitable age, including infant, juvenile, adolescent, adult, and geriatric subjects.

The term “cancer” or “tumor” refers to the presence of cells possessing characteristics typical of cancer-causing cells, such as uncontrolled proliferation, immortality, metastatic potential, rapid growth and proliferation rate, and certain characteristic morphological features. Cancer cells are often in the form of a solid tumor, which is detectable on the basis of tumor mass, e.g., by procedures such as CAT scan, MR imaging, X-ray, ultrasound or palpation, and/or which is detectable because of the expression of one or more cancer-specific antigens in a sample obtainable from a patient. In some embodiments, a solid tumor does not need to have measurable dimensions. Cancer cells may also in the form of a liquid tumor, which cancer cells may exist alone or disseminated within an animal. As used herein, the terms “disseminated tumor” and “liquid tumor” are used interchangeably, and include, without limitation, leukemia and lymphoma and other blood cell cancers.

The terms “cancer recurrence” and “cancer relapse” are used interchangeably and refer to the return of a sign, symptom or disease after a remission. The recurrent cancer cells may re-appear in the same site of the primary tumor or in another location, such as in secondary cancer. The cancer cells may re-appear in the same diseased form as the primary cancer or a different diseased form. For example, in some embodiments, a primary cancer is a solid tumor, and the recurrent cancer is a liquid tumor. In other embodiments, a primary cancer is a liquid tumor, and the recurrent cancer is a solid tumor. In yet other embodiments, the primary cancer and the recurrent cancer are both solid tumors, or both liquid tumors. In some embodiments, the recurrent tumor expresses at least one tumor-associated antigen that is also expressed by the primary tumor.

The term “refractory cancer” as used herein refers to a cancer that does not respond to a treatment, for example, a cancer that is resistant at the beginning of treatment (e.g., treatment with an immunotherapy) or a cancer that may become resistant during treatment. The terms “respond,” “response” or “responsiveness” refer to an anti-cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

As used herein, cancers include, but are not limited to, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, gastric cancer, cervical cancer, uterine or endometrial cancer, cancer of the oral cavity or pharynx, cancer of the nasopharynx, cancer of the sinuses, liver cancer, kidney cancer, testicular cancer, neuroendocrine cancer, biliary tract cancer, small bowel or appendix cancer, salivary gland cancer, thyroid gland cancer, adrenal gland cancer, osteosarcoma, chondrosarcoma, cancer of hematologic tissues, B cell cancer, e.g., multiple myeloma, Waldenstrom's macroglobulinemia, the heavy chain diseases, such as, for example, alpha chain disease, gamma chain disease, and mu chain disease, benign monoclonal qammopathy, and immunocytic amyloidosis, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, sominoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., high grade serous ovarian carcinoma), or breast carcinoma. The epithelial cancers may be characterized in various other ways including, but not limited to, serous, endometrioid, mucinous, clear cell, Brenner, or undifferentiated.

The term “cancer therapy” or “cancer therapeutic agent” as used herein, refers to those therapies or agents that can exert anti-tumor effect or have an anti-tumor activity. Such anti-tumor effect or anti-tumor activity can be exhibited as a reduction in the rate of tumor cell proliferation, viability, or metastatic activity. A possible way of showing anti-tumor activity is to show a decline in growth rate of abnormal cells that arises during therapy or tumor size stability or reduction. Such activity can be assessed using accepted in vitro or in vivo tumor models, including but not limited to xenograft models, allograft models, MMTV models, and other known models known in the art to investigate anti-tumor activity.

The terms “treat,” “treating,” and “treatment” are meant to include alleviating or abrogating a condition, disorder, or disease, or one or more of the symptoms associated with the condition, disorder, or disease; or alleviating or eradicating the cause(s) of the condition, disorder, or disease itself. In some embodiments, treatment of a cancer results in a complete response (CR) or partial response (PR). In some embodiments, treatment of a cancer results in a CR, PR, or stable disease (SD). Exemplary criteria for CR, PR, and SD are provided herein.

The terms “prevent,” “preventing,” and “prevention” are meant to include a method of delaying and/or precluding the onset of a condition, disorder, or disease, and/or its attendant symptoms; barring a subject from acquiring a condition, disorder, or disease; or reducing a subject's risk of acquiring a condition, disorder, or disease.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

The term “Q-tag,” as used herein, refers to a portion of a polypeptide containing glutamine residue that, upon transglutaminase-mediated reaction with a compound containing —NH₂ amine, provides a conjugate containing the portion of polypeptide, in which the glutamine residue includes a side chain modified to include the amide bonded to the compound. Q-tags are known in the art. Non-limiting examples of Q-tags are LLQGG (SEQ ID NO:12), GGGLLQGG (SEQ ID NO:13), RPQGF (SEQ ID NO: 1), and RPQGFGPP (SEQ ID NO:2).

The Q tag may be attached to the heavy chain of the antibody, to the light chain of the antibody, or within the Fc domain of the antibody. In some cases, the Q tag is naturally occurring. For example, mutation of N297 to N297A exposes Q295 of the antibody, where the conjugation could occur (numbering according to EU index, e.g., as listed in Edelman, G. M. et al., Proc. Natl. Acad. USA, 63, 78-85 (1969) and Kabat, E. A. et al., Sequences of proteins of immunological interest. 5th Edition—US Department of Health and Human Services, NIH publication no 91-3242, pp 662,680,689 (1991)).

### II. Methods of Treating Cancer

Provided herein are methods of treating cancer in an individual comprising administering a compound of Formula I to achieve an effective therapeutic dose while minimizing side effects. The compound of Formula I is a conjugate comprising an anti-CD22 antibody and an immunomodulating oligonucleotide (e.g., CpG oligonucleotide), wherein the Ab comprises two antibody light chains, two antibody heavy chains, and two Q-tag peptides (Q); wherein each of the two Q-tag peptides comprises the amino acid sequence RPQGFGPP (SEQ ID NO:2); wherein one Q-tag peptide is linked to the C-terminus of each of the two antibody heavy chains; wherein one of the two Q-tag peptides is linked to the immunomodulating oligonucleotide via an amide bond with the glutamine residue of the Q-tag peptide and a linker (L) as shown in Formula (A):

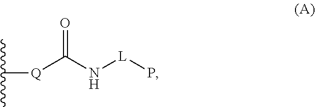

wherein [symbol] indicates the point of attachment of Q to the antibody (Ab);
wherein each heavy chain of the Ab comprises a CDR1 comprising the amino acid sequence of GFAFSIYD (SEQ ID NO:6), a CDR2 comprising the amino acid sequence of ISSGGGTT (SEQ ID NO:7), and a CDR3 comprising the amino acid sequence of ARHSGYGTHWGVLFAY (SEQ ID NO:8), and each light chain of the Ab comprises a CDR1 comprising the amino acid sequence of QDIHGY (SEQ ID NO:9), a CDR2 comprising the amino acid sequence of YTS, and a CDR3 comprising QQGSTLPWT (SEQ ID NO:11);
wherein the linker L is

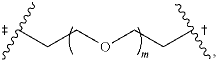

wherein m is 24, and [symbol] indicates the point of attachment to the oligonucleotide P, and [symbol] indicates the point of attachment to the glutamine residue of the Q-tag; and
wherein the oligonucleotide P comprises the structure:

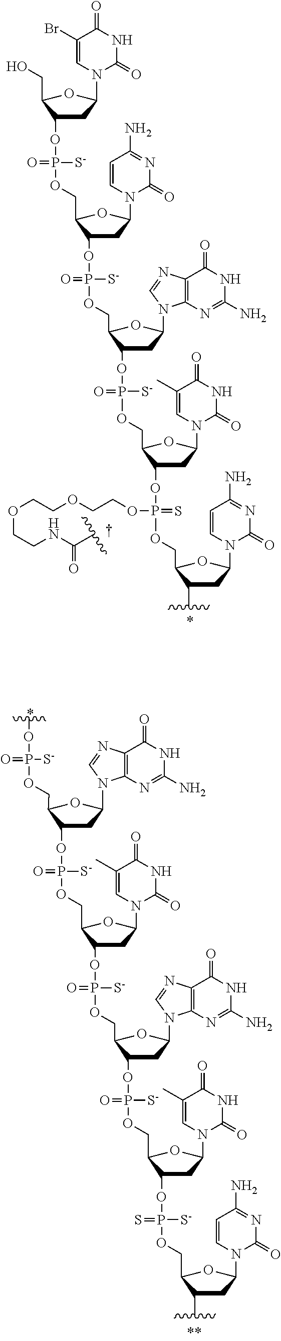

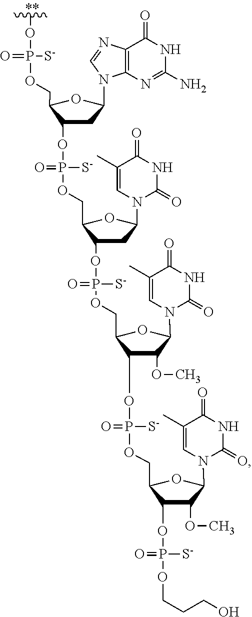

or a pharmaceutically acceptable salt thereof,
   wherein [symbol] * and [symbol] ** indicate the points of attachment within the oligonucleotide, wherein [symbol] indicates the point of attachment to the linker L. In some embodiments, the compound of Formula I is administered in a dosing range between 3 mg/kg and 30 mg/kg. In some embodiments, the dose of Formula I administered is 3 mg/kg, 6 mg/kg, 12 mg/kg, 18 mg/kg, 24 mg/kg or 30 mg/kg. In some embodiments, the dose of Formula I administered is between about 3 mg/kg and about 6 mg/kg. In some embodiments, the dose of Formula I administered is between about 6 mg/kg and about 12 mg/kg. In some embodiments, the dose of Formula I administered is between about 12 mg/kg and about 18 mg/kg. In some embodiments, the dose of Formula I administered is between about 18 mg/kg and about 24 mg/kg. In some embodiments, the dose of Formula I administered is between about 24 mg/kg and about 30 mg/kg.

In some embodiments, the compound of Formula I is administered to an individual in combination with a second therapeutic agent. In some embodiments, the second therapeutic agent is an anti-PD-1 antibody. In some embodiments, the anti-PD-1 antibody is pembrolizumab. In some embodiments, the pembrolizumab is administered Q3W (once every three weeks). In some embodiments, the pembrolizumab is administered Q6W (once every six weeks). In some embodiments, the pembrolizumab and the compound of Formula I are administered Q3W (once every three weeks). In some embodiment, pembrolizumab is administered and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg). In some embodiments, the compound of Formula I is administered Q3W and the pembrolizumab is administered Q6W.

In some embodiments, the method of treating cancer includes administering the compound of Formula I or administering the compound of Formula I and an anti-PD-1 antibody such as pembrolizumab to an individual having a solid tumor. In some aspects, the solid tumor is an advanced or metastatic solid tumor. In some embodiments, the solid tumor is a histologically or cytologically-documented solid tumor that is inoperable, locally advanced, metastatic, or recurrent. In some embodiments, compound of Formula I or the compound of Formula I and an anti-PD-1 antibody such as pembrolizumab is administered to an individual that has a solid tumor and the cancer in the individual has progressed on or the individual is intolerant to standard therapy, or has a cancer for which no standard therapy is available. In some embodiments, the individual has a progressed on or the cancer has recurred after prior treatment with an immune-oncology agent. Immuno-oncology agents modulate the immune system to attack cancer cells and such agents include, but are not limited to inhibitors of PD-1, PD-L1, LAG3 and CTLA-4, such as a small molecule or antibody that inhibits PD-1, PD-L1, LAG3 or CTLA-4. In some aspects, the prior treatment is treatment with an anti-PD-1 antibody and/or an anti-CTLA4 antibody. In some embodiments, the cancer to be treated is melanoma, cutaneous melanoma, uveal melanoma, acral melanoma, breast cancer, ovarian cancer, colorectal cancer, non-small cell lung cancer (NSCLC), cutaneous squamous cell cancer, renal cell carcinoma (RCC), head and neck squamous cell carcinoma (HNSCC), Merkle cell carcinoma, hepatocellular carcinoma (HCC), cervical squamous cell carcinoma, urothelial carcinoma, endometrial carcinoma, gastro-esophageal adenocarcinoma, or cholangiocarcinoma (including internal and external cholangiocarcinoma (cancers of the internal and external bile ducts) and gall bladder cancers). In some embodiments, the cancer to be treated is triple negative breast cancer, melanoma, ovarian cancer or colon cancer. In some embodiments, the cancer to be treated is cholangiocarcinoma. In some embodiments, the cancer to be treated is melanoma. In some embodiments, the cancer to be treated is cutaneous melanoma.

In some embodiments, the cancer to be treated with the compound of Formula I or the compound of Formula I and an anti-PD-1 antibody such as pembrolizumab is melanoma, cutaneous melanoma. uveal melanoma or acral melanoma. In some embodiments, tumor cell(s) of the cutaneous melanoma have a genetic mutation, such as a mutation in one or more of BRAF, NTRK and KIT. In some embodiments, tumor cell(s) of the cutaneous melanoma do not have a genetic mutation. In some embodiments, the cancer to be treated with the compound of Formula I or the compound of Formula I and an anti-PD-1 antibody such as pembrolizumab is a biliary tract cancer, such as intra-or extrahepatic cholangiocarcinoma, or a gall bladder cancer. In some embodiments, the cancer to be treated with the compound of Formula I or the compound of Formula I and an anti-PD-1 antibody such as pembrolizumab is an immunogenic solid tumor. In some embodiments, the immunogenic solid tumor is a gastric cancer, a cancer of the stomach and gastroesophageal junction, a non-small cell lung cancer (NSCLC), renal cell carcinoma (RCC), urothelial cancer, cutaneous squamous cell carcinoma, Merkel cell carcinoma or a tumor with one or more of known microsatellite instable (MSI-H), mismatch repair (MMMR) deficient, and tumor mutation burden high (TMB-H, >10 mut/Mb). In some embodiments, the cancer to be treated with the compound of Formula I or the compound of Formula I and an anti-PD-1 antibody such as pembrolizumab is a solid tumor or tumor cells with one or more genetic mutations, such as a mutation in one or more of HER2, BRAF, FGFR, IDH1, NTRK, RET, KIT, ROS1, NRAS, ALK, MET, and KRAS.

In some embodiments, the compound of Formula I is administered to an individual at dose between 3 mg/kg and 30 mg/kg (e.g., 3 mg/kg, 6 mg/kg, 12 mg/kg, 18 mg/kg, 24 mg/kg or 30 mg/kg, or between about 3 mg/kg and about 6 mg/kg, between about 6 mg/kg and about 12 mg/kg, between about 12 mg/kg and about 18 mg/kg, between about 18 mg/kg and about 24 mg/kg, or between about 24 mg/kg and about 30 mg/kg) and the dose is administered Q2W (biweekly), or about once every 14 days. In some embodiments, the compound of Formula I is administered to an individual at dose between 3 mg/kg and 30 mg/kg (e.g., 3 mg/kg, 6 mg/kg, 12 mg/kg, 18 mg/kg, 24 mg/kg or 30 mg/kg, or between about 3 mg/kg and about 6 mg/kg, between about 6 mg/kg and about 12 mg/kg, between about 12 mg/kg and about 18 mg/kg, between about 18 mg/kg and about 24 mg/kg, or between about 24 mg/kg and about 30 mg/kg) and the dose is administered Q1W (weekly). In some embodiments, the compound of Formula I is administered to an individual at dose between 3 mg/kg and 30 mg/kg (e.g., 3 mg/kg, 6 mg/kg, 12 mg/kg, 18 mg/kg, 24 mg/kg or 30 mg/kg, or between about 3 mg/kg and about 6 mg/kg, between about 6 mg/kg and about 12 mg/kg, between about 12 mg/kg and about 18 mg/kg, between about 18 mg/kg and about 24 mg/kg, or between about 24 mg/kg and about 30 mg/kg) and the dose is Q3W (every 3 weeks), or about once every 21 days. In some embodiments, the compound of Formula I is administered to an individual at dose between 3 mg/kg and 30 mg/kg (e.g., 3 mg/kg, 6 mg/kg, 12 mg/kg, 18 mg/kg, 24 mg/kg or 30 mg/kg, or between about 3 mg/kg and about 6 mg/kg, between about 6 mg/kg and about 12 mg/kg, between about 12 mg/kg and about 18 mg/kg, between about 18 mg/kg and about 24 mg/kg, or between about 24 mg/kg and about 30 mg/kg) and the dose is administered Q4W (once every 4 weeks), about once every 28 days, or about once a month. In some embodiments, the compound of Formula I is administered to an individual at dose between 3 mg/kg and 30 mg/kg (e.g., 3 mg/kg, 6 mg/kg, 12 mg/kg, 18 mg/kg, 24 mg/kg or 30 mg/kg, or between about 3 mg/kg and about 6 mg/kg, between about 6 mg/kg and about 12 mg/kg, between about 12 mg/kg and about 18 mg/kg, between about 18 mg/kg and about 24 mg/kg, or between about 24 mg/kg and about 30 mg/kg) and the dose is administered Q1W (weekly), or about once every 7 days. In some embodiments, the dose (e.g., administered Q2W, Q3W, Q4W, or Q1W) is administered once, twice, 3 times, 4 times, 5 times or more than 5 times to an individual during a course of treatment. In some embodiments, the compound of Formula I is administered at the dose and doing schedule described herein to the individual intravenously (IV). In some embodiments, the compound of Formula I at the dose and doing schedule described herein is administered in a formulation comprising the conjugate at a concentration of 30 mg/mL; 20 mM citrate; 150 mM L-arginine; 50 mM NaCl; and 0.02% polysorbate 80 (w/v); wherein the formulation is at pH 6.5.

In some embodiments, the compound of Formula I is administered to an individual in combination with a second therapeutic agent. In some embodiments, the second therapeutic agent is an anti-PD-1 antibody. In some embodiments, the anti-PD-1 antibody is pembrolizumab. In some embodiments, the pembrolizumab is administered Q3W (once every three weeks). In some embodiments, the pembrolizumab and the compound of Formula I are administered Q3W (once every three weeks). In some embodiments, the pembrolizumab is administered Q3W (once every three weeks) and the compound of Formula I is administered Q1W (weekly). In some embodiments, the pembrolizumab is administered Q3W (once every three weeks) and the compound of Formula I is administered Q2W (biweekly). In some embodiments, the pembrolizumab is administered Q3W (once every three weeks) and the compound of Formula I is administered Q4W (once every 4 weeks), about once every 28 days, or about once a month. In some embodiments, the compound of Formula I is administered Q1W, Q2W, Q3Q or Q4W and the pembrolizumab is administered Q6W.

In some embodiments, the pembrolizumab is administered Q3W (once every three weeks) at a dose of 200 mg and the compound of Formula I are administered Q3W (once every three weeks). In some embodiments, the pembrolizumab is administered Q3W (once every three weeks) at a dose of 200 mg and the compound of Formula I is administered Q1W (weekly). In some embodiments, the pembrolizumab is administered Q3W (once every three weeks) at a dose of 200 mg and the compound of Formula I is administered Q2W (biweekly). In some embodiments, the pembrolizumab is administered Q3W (once every three weeks) at a dose of 200 mg and the compound of Formula I is administered Q4W (once every 4 weeks), about once every 28 days, or about once a month. In some embodiments, the pembrolizumab is administered Q6W (once every six weeks) at a dose of 400 mg and the compound of Formula I is administered Q1W, Q2W, Q3Q or Q4W.

In some embodiment, pembrolizumab is administered and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg).

In some embodiment, pembrolizumab is administered and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q3W and the compound of Formula I is administered Q3W. In some embodiment, pembrolizumab is administered and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q3W and the compound of Formula I is administered Q1W. In some embodiment, pembrolizumab is administered and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q3W and the compound of Formula I is administered Q2W. In some embodiment, pembrolizumab is administered and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q3W and the compound of Formula I is administered Q4W.

In some embodiment, pembrolizumab is administered and the compound of Formula I is administered, pembrolizumab is administered at a dose of 200 mg and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg). In some embodiment, pembrolizumab is administered and the compound of Formula I is administered, pembrolizumab is administered at a dose of 400 mg and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg).

In some embodiment, pembrolizumab is administered at a dose of 200 mg and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q3W and the compound of Formula I is administered Q3W. In some embodiment, pembrolizumab is administered at a dose of 200 mg and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q3W and the compound of Formula I is administered Q1W. In some embodiment, pembrolizumab is administered at a dose of 200 mg and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q3W and the compound of Formula I is administered Q2W. In some embodiment, pembrolizumab is administered at a dose of 200 mg and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg) and about 6 mg/kg and pembrolizumab is administered Q3W and the compound of Formula I is administered Q4W.

In some embodiment, pembrolizumab is administered at a dose of 400 mg and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q6W and the compound of Formula I is administered Q1W. In some embodiment, pembrolizumab is administered at a dose of 400 mg and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q6W and the compound of Formula I is administered Q1W. In some embodiment, pembrolizumab is administered at a dose of 400 mg and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg and about 6 mg/kg) and pembrolizumab is administered Q6W and the compound of Formula I is administered Q2W. In some embodiment, pembrolizumab is administered at a dose of 400 mg and the compound of Formula I is administered, and the compound of Formula I is administered at dose between 0.3 mg/kg and 6 mg/kg (e.g., 0.3 mg/kg, 1 mg/kg, 3 mg/kg, or 6 mg/kg, or between about 0.3 mg/kg and about 1 mg/kg, between about 1 mg/kg and about 3 mg/kg or between about 3 mg/kg) and about 6 mg/kg and pembrolizumab is administered Q6W and the compound of Formula I is administered Q4W.

In some embodiments, the dose of the compound of Formula I (e.g., administered Q2W, Q3W, Q4W, or Q1W) is administered once, twice, 3 times, 4 times, 5 times or more than 5 times to an individual during a course of treatment. In some embodiments, the dose of the compound of Formula I (e.g., administered Q2W, Q3W, Q4W, or Q1W) is administered once, twice, 3 times, 4 times, 5 times or more than 5 times to an individual during a course of treatment and an anti-PD-1 antibody such as pembrolizumab (e.g., administered Q3W) is administered once, twice, 3 times, 4 times, 5 times or more than 5 times to an individual during a course of treatment. In some embodiments, the compound of Formula I is administered at the dose and dosing schedule described herein to the individual intravenously (IV).

In some embodiments, the compound of Formula I is administered to an individual at dose between 3 mg/kg and 30 mg/kg (e.g., 3 mg/kg, 6 mg/kg, 12 mg/kg, 18 mg/kg, 24 mg/kg or 30 mg/kg, or between about 3 mg/kg and about 6 mg/kg, between about 6 mg/kg and about 12 mg/kg, between about 12 mg/kg and about 18 mg/kg, between about 18 mg/kg and about 24 mg/kg, or between about 24 mg/kg and about 30 mg/kg) and the dose is administered on a schedule of dosing Q2W, Q3W, Q4W or Q1W and treatment according to a method of the present disclosure results in a therapeutic response in the individual. In some embodiments, the compound of Formula I is administered to an individual at dose between 0.3 mg/kg and 6 mg/kg (e.g., 0.3 mg/kg, 1 mg/kg, 3 mg/kg, 6 mg/kg, or between about 0.3 mg/kg and about 1 mg/kg, between about 1 mg/kg and about 3 mg/kg, or between about 3 mg/kg and about 6 mg/kg) and the dose is administered on a schedule of dosing Q2W, Q3W, Q4W or Q1W, and additionally an anti-PD-1 antibody such as pembrolizumab (e.g., administered Q3W at a dose of 200 mg) is administered and treatment according to a method of the present disclosure results in a therapeutic response in the individual. In some embodiments, the therapeutic response is a complete response (CR), a partial response (PR) or stable disease (SD) in the individual. In some embodiments, treatment according to a method of the present disclosure results in a complete response (CR) in the individual. In some embodiments, a CR refers to disappearance of all non-nodal target lesions and a reduction in short axis to <10 mm of any pathological lymph nodes assigned as target lesions. In some embodiments, treatment according to a method of the present disclosure results in a partial response (PR) in the individual. In some embodiments, a PR refers to at least a 30% decrease in the sum of diameter of all target lesions, taking as reference the baseline sum of diameters. In some embodiments, treatment according to a method of the present disclosure results in stable disease (SD) in the individual. In some embodiments, SD refers to insufficient target lesion shrinkage to qualify for PR or CR (e.g., as described above) and insufficient increase in target lesion(s) to qualify for progressive disease (PD). In some embodiments, PD refers to at least a 20% increase in the sum of diameter of all measured target lesions, taking as reference the smallest sum of diameter of all target lesions recorded at or after baseline, as well as the sum demonstrating an absolute increase of at least 5 mm.

In some embodiments, the compound of Formula I is administered to an individual at dose between 3 mg/kg and 30 mg/kg (e.g., 3 mg/kg, 6 mg/kg, 12 mg/kg, 18 mg/kg, 24 mg/kg or 30 mg/kg, or between about 3 mg/kg and about 6 mg/kg, between about 6 mg/kg and about 12 mg/kg, between about 12 mg/kg and about 18 mg/kg, between about 18 mg/kg and about 24 mg/kg, or between about 24 mg/kg and about 30 mg/kg) and the dose is administered on a schedule of dosing Q2W, Q3W, Q4W or Q1W and is used to treat a cancer, such as a solid tumor. In some embodiments, the compound of Formula I is administered to an individual at dose between 0.3 mg/kg and 6 mg/kg (e.g., 0.3 mg/kg, 1 mg/kg, 3 mg/kg, 6 mg/kg, or between about 0.3 mg/kg and about 1 mg/kg, between about 1 mg/kg and about 3 mg/kg, or between about 3 mg/kg and about 6 mg/kg) and the dose is administered on a schedule of dosing Q2W, Q3W, Q4W or Q1W, and additionally an anti-PD-1 antibody such as pembrolizumab (e.g., administered Q3W at a dose of 200 mg) is administered and is used to treat a cancer, such as a solid tumor. In some embodiments, the solid tumor to be treated is inoperable, locally advanced, metastatic, and/or recurrent. In some embodiments, a locally advanced or advanced cancer refers to a cancer that has spread only to nearby tissues or lymph nodes. For example, for locally advanced breast cancer, cancers may involve the skin of the breast or the underlying muscles of the chest or involve multiple local lymph nodes (those located in the arm pit or the soft tissues above and below the collarbone). For example, for lung cancer; locally advanced lung cancer is when the cancer has grown into an airway, the chest wall or the membranes that surround the lung (the pleura). In some embodiments, a metastatic cancer refers to a cancer that has spread to distant part(s) of the body, such as to the bones, lung, brain and liver; for example, a metastatic lung cancer, where the cancer has spread to the brain, bones, liver, adrenal glands, other parts of the lung or the other lung. In some embodiments, a cancer (e.g., solid tumor) to be treated by the methods of the present disclosure is histologically and/or cytologically documented.

Certain aspects of the present disclosure relate to CD22 and anti-CD22 antibodies. In some embodiments, CD22 refers to human CD22, and the antibodies bind human CD22. CD22 is also known as Siglec-2, and CD22 gene and polypeptide sequences (e.g., human gene and polypeptide sequences) are known in the art. See, e.g., NCBI Gene ID No. 933 and NCBI Ref Seq. Accession No. NP_001172028. Any of the anti-CD22 antibodies provided herein may find use in the conjugates and methods of the present disclosure.

In some embodiments, the anti-CD22 antibody portion of the compound of Formula I comprises a VH domain comprising QVQLLESGGGVVQPGGSLRLSCAASGFAFSIYDMNWVRQAPGKGLEWVSAISSGGG TTYYADSVKGRFTISRDNAKNSLYLQMNSLRAEDTAVYYCARHSGYGTHWGVLFA YWGRGTLVTVSS (SEQ ID NO:3). In some embodiments, the anti-CD22 antibody portion of the compound of Formula I comprises a VL domain comprising the sequence of DIQMTQSPSSLSASVGDRVTITCRASQDIHGYLNWYQQKPGKAPKLLIYYTSILHSGV PSRFSGSGSGTDFTLTISSLQPEDFATYFCQQGSTLPWTFGQGTKLEIK (SEQ ID NO: 4). In some embodiments, the anti-CD22 antibody portion of the compound of Formula I comprises a VH domain comprising the sequence of SEQ ID NO:3 and a VL domain comprising the sequence of SEQ ID NO:4. In some embodiments, the anti-CD22 antibody portion of the compound of Formula I comprises a VH domain comprising the sequence of SEQ ID NO:3, a heavy chain constant region comprising

(SEQ ID NO: 5)
ASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKS
CDKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVF
SCSVMHEALHNHYTQKSLSLSPG

and a VL domain comprising the sequence of SEQ ID NO:4. In some embodiments, the anti-CD22 antibody portion of the compound of Formula I comprises a heavy chain and a Q-tag on the C-terminus of the heavy chain comprising the sequence of QVQLLESGGGVVQPGGSLRLSCAASGFAFSIYDMNWVRQAPGKGLEWVSAISSGGG TTYYADSVKGRFTISRDNAKNSLYLQMNSLRAEDTAVYYCARHSGYGTHWGVLFA YWGRGTLVTVSSASTKGPSVFPLAPSSKSTSGGTAALGCLVKDYFPEPVTVSWNSGA LTSGVHTFPAVLQSSGLYSLSSVVTVPSSSLGTQTYICNVNHKPSNTKVDKKVEPKSC DKTHTCPPCPAPEAAGAPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPI EKTISKAKGQPREPQVYTLPPSREEMTKNQVSLTCLVKGFYPSDIAVEWESNGQPEN NYKTTPPVLDSDGSFFLYSKLTVDKSRWQQGNVFSCSVMHEALHNHYTQKSLSLSP GRPQGFGPP (SEQ ID NO: 14). In some embodiments, the anti-CD22 antibody portion of the compound of Formula I comprises a light chain comprising the sequence of DIQMTQSPSSL SASVGDRVTITCRASQDIHGYLNWYQQKPGKAPKLLIYYTSILHSGV PSRFSGSGSGTDFTLTISSLQPEDFATYFCQQGSTLPWTFGQGTKLEIKRTVAAPSVFIF PPSDEQLKSGTASVVCLLNNFYPREAKVQWKVDNALQ SGNSQESVTEQDSKDSTYS LSSTLTLSKADYEKHKVYACEVTHQGLSSPVTKSFNRGEC (SEQ ID NO: 15). In some embodiments, the anti-CD22 antibody portion of the compound of Formula I comprises a heavy chain comprising the sequence of SEQ ID NO: 14 and a Q-tag on the C-terminus of the heavy chain and a light chain comprising the sequence of SEQ ID NO: 15.

### III. KITS

Also provided herein is a kit comprising a conjugate as described above for use in any of methods described herein.

In another aspect, the kit further comprises a package insert including, without limitation, appropriate instructions for preparation and administration of the formulation, side effects of the formulation, and any other relevant information. The instructions may be in any suitable format, including, but not limited to, printed matter, videotape, computer readable disk, optical disc or directions to internet-based instructions.

In another aspect, kits for treating an individual who suffers from or is susceptible to the conditions described herein are provided, comprising a first container comprising a dosage amount of a composition or formulation as disclosed herein, and a package insert for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the formulation to be administered to the individual.

In another aspect, kits may also be provided that contain sufficient dosages of the compositions described herein (including pharmaceutical compositions thereof) to provide effective treatment for an individual for an extended period, such as 1-3 days, 1-5 days, a week, 2 weeks, 3, weeks, 4 weeks, 6 weeks, 8 weeks, 1 cycle, 2 cycles, 3 cycles, 4 cycles, 5 cycles, 6 cycles, 7 cycles, 8 cycles or more.

In some embodiments, the kits may also include multiple doses and may be packaged in quantities sufficient for storage and use in pharmacies, for example, hospital pharmacies and compounding pharmacies. In certain embodiments the kits may include a dosage amount of at least one composition as disclosed herein.

### EXAMPLES

The presently disclosed subject matter will be better understood by reference to the following Examples, which are provided as exemplary of the invention, and not by way of limitation.

### Example 1: An Open-Label, Dose Escalation Study of the Compound of Formula I in Patients with Advanced or Metastatic Solid Tumors

The compound of Formula I (also referred to herein as “Compound 1”) was administered to patients every 2 weeks (Q2W) by intravenous infusion over approximately 90 min, starting at 0.1 mg/kg, followed by dose cohorts at 0.3 mg/kg, 1.0 mg/kg, and 3.0 mg/kg. During the first week after receiving the first dose, patients were monitored for daily temperature and any CRS symptoms. Patients were monitored for 28 days to assure no dose limiting toxicities (DLT) arose, with a DLT target rate of 25%. All patients were also monitored for late DLTs during Days 29-60. In the absence of any DLTs or ≥Grade 2 cytokine release syndrome (CRS), the next group of patients were escalated to the next dose in the series.

Patient baseline characteristics for doses between 0.1-3 mg/kg: 18 total subjects (6 female, 12 male); median age 57.0 (range 41-76) years old; median prior therapies 3 (range 1-7). Median duration of treatment 1.4 months, range 1-11 months. Table 1 shows the tumor types of subjects in the study for doses between 0.1-3 mg/kg. At the 3.0 mg/kg cohort, one patient exhibited a partial response (PR) and in two other patients a DLT or ≥Grade 2 CRS was observed.

TABLE 1
Subject baseline tumor types

| | Number of subjects (% |
|---|---|
| Primary Cancer Diagnosis | total) |
| Colorectal carcinoma | 9 (50%) |
| Ovarian carcinoma | 2 (11%) |
| Cutaneous melanoma | 2 (11%) |
| Head and neck squamous cell carcinoma | 2 (11%) |
| Cholangiocarcinoma | 2 (11%) |
| Gastro-esophageal carcinoma | 1 (6%) |

This study included a screening period, a treatment period, and a follow-up period. All patients completed up to 28 days of screening. During the treatment period, patients received the compound of Formula I biweekly (Q2W) until treatment discontinuation criteria were met. Patients then entered a follow-up period, to be continued for up to 2 years or until study discontinuation criteria are met. Following the review of pharmacokinetic data from the first 4 dosing cohorts (shown in Example 5) and CD22 internalization data (shown in Example 2), additional dosing cohorts were added to include the following doses: 6 mg/kg, and 12 mg/kg to be administered every 4 weeks, and dosing cohorts may also include 18 mg/kg, 24 mg/kg and 30 mg/kg. Compound 1 does not block its target (CD22), and as such, the dosing of Compound 1 may have notable differences from dosing a drug, such as an antibody drug conjugate that works by blocking its target.

Inclusion criteria included:
(a) Age (≥18 years old);
(b) Sex (all);
(c) Histologically or cytologically-documented solid tumors that are inoperable, locally advanced, metastatic, or recurrent of the following cancers:
   (i) Cutaneous melanoma;
   (ii) Breast cancer;
   (iii) Ovarian cancer;
   (iv) Colorectal cancer;
   (v) Non-small cell lung cancer (NSCLC);
   (vi) Renal cell carcinoma (RCC);
   (vii) Head and neck squamous cell carcinoma (HNSCC):
   (viii) Merkel cell carcinoma;
   (ix) Hepatocellular carcinoma (HCC);
   (x) Cervical squamous cell carcinoma;
   (xi) Urothelial carcinoma;
   (xii) Endometrial carcinoma
   (xiii) Gastro-esophageal adenocarcinoma; and
   (xiv) Cholangiocarcinoma;
(d) Patients must have at least one measurable lesion, as defined by RECIST v1.1
(e) Adult male or female patients ≥18 years of age on day of signing informed consent.
(f) Eastern Cooperative Oncology Group (ECOG) performance status (PS) 0 or 1;
(g) Demonstration of adequate organ function.

Exclusion criteria included:
(a) Prior history of or active malignant disease other than that being treated in this study; (b) Known brain metastases or cranial epidural disease;
(c) Receiving chronic systemic steroid therapy (>10 mg/day prednisone or equivalent) or any immunosuppressive therapy within 7 days prior to the first dose of study drug;
(d) Active autoimmune disease requiring systemic treatment in the past 2 years;
(e) History of Grade 3 or higher immune mediated adverse events that were considered drug-related to prior immunotherapy;
(f) Infection with HIV-1 or HIV-2, unless HIV is well-controlled and viral load is undetectable; infection with hepatitis B unless disease is undetectable; infection with hepatitis C unless disease is well-controlled and viral load is minimal;
(g) Active infection requiring systemic therapy;
(h) Significant cardiovascular disease within the last 6 months;
(i) Receipt of any type of systemic anti-cancer therapy within 4 weeks of the first dose of study drug;
(j) Current or previous participation in an interventional clinical trial with an investigational compound or device within 4 weeks of the first dose of study drug;
(k) Radiation for bone metastasis within 2 weeks or any other radiation therapy within 4 weeks of the first dose of study drug;
(l) Major surgery within 2 weeks of the first dose of study drug;
(m) Receipt of any live vaccines within 4 weeks or any other vaccines within 2 days of the first dose of study drug;
(n) Any prior treatment with TLR9 agonists or anti-CD22 antibodies;
(o) A known hypersensitivity to the components of the study therapy or its analogs;
(p) A history or current evidence of any condition therapy, lab abnormality, or other circumstance exposing the subject to risk by participating in the trial, confound the results of the trial, or interfere with the patient's participation for the full duration of the trial; and
(q) Known psychiatric or substance abuse disorders that would interfere with cooperation with the requirements of the trial.

The primary outcome measure included an analysis of safety and tolerability of increasing dose levels of the anti-CD22-CpG conjugate in order to identify the recommended dose for a Phase 2 expansion study. This outcome measure included the assessment of 28-day dose limiting toxicities (DLTs), adverse events (AEs), and laboratory abnormalities. AEs are characterized by type, frequency, and severity per National Cancer Institute Common Terminology Criteria for Adverse Events (NCI CTCAE) v 5.0, and laboratory abnormalities are graded by CTCAE v 5.0.

Secondary outcome measures included the following:
(a) Characterization of single and multiple dose pharmacokinetics of the anti-CD22-CpG conjugate, including pharmacokinetic parameters such as serum concentration (Cₘₐₓ), shortest time to peak plasma concentration (Tₘₐₓ), area under curve (AUC), drug clearance (CL), and half-life (t_{1/2}) as data allow;
(b) Evaluation of immunogenicity of the anti-CD22-CpG conjugate and the incidence of antidrug antibodies against this anti-CD22-CpG conjugate; and
(c) Evaluation of preliminary anti-tumor activity as assessed for all cohorts by best overall response rate (ORR) per RECIST v1.1 and iRECIST and by duration of response (DOR) and the clinical benefit rate, and as assessed for ovarian cancer patients by combined RECIST v1.1 and Gynecologic Cancer Intergroup (GCIG) CA-125 ORR and DOR criteria. Response criteria are shown in Tables E1-E2C below.

Exploratory outcome measures included the following:
(a) Characterization of other efficacy outcomes, such as progression-free survival (PFS) and overall survival (OS);
(b) Characterization of the degree of target engagement by the anti-CD22-CpG conjugate via measurement CD22 expression on B cells in peripheral blood;
(c) Exploration of the impact of the anti-CD22-CpG conjugate on systemic soluble immune factors, including pre- and post-dose quantification of soluble immune factors in serum;
(d) Assessment of the pharmacodynamics effect of the anti-CD22-CpG conjugate on immune cell markers, including levels of phenotypic markers on B cells and lymphocyte counts in peripheral blood; and
(e) Exploration of the impact of the anti-CD22-CpG conjugate on pharmacodynamics markers in tumor tissues, including pre- and post-dose levels of pharmacodynamics markers that include but are not limited to (i) tumor mutational burden (TMB) and (ii) PD-L1 and other immune modulatory markers.

TABLE E1
Response criteria for the evaluation of target lesions.

| Response | |
|---|---|
| Criteria | Evaluation of Target Lesions |
| Complete | Disappearance of all non-nodal target lesions. In |
| Response | addition, any pathological lymph nodes assigned |
| (CR) | as target lesions must have a reduction in short |
| | axis to <10 mm. ¹ |
| Partial | At least a 30% decrease in the sum of diameter |
| Response | of all target lesions, taking as reference the baseline |
| (PR) | sum of diameters. |
| Progressive | At least a 20% increase in the sum of diameter of all |
| Disease | measured target lesions, taking as reference the smallest |
| (PD) | sum of diameter of all target lesions recorded at or after |
| | baseline. In addition to the relative increase of 20%, the |
| | sum must also demonstrate an absolute increase of at |
| | least 5 mm. ² |
| Stable Disease | Neither sufficient shrinkage to qualify for PR or CR |
| (SD) | nor an increase in lesions which would qualify for |
| | PD. |
| Unknown | Progression has not been documented and one or |
| (UNK) | more target lesions have not been assessed or have |
| | been assessed using a different method than baseline. |

¹ SOD for CR may not be zero when nodal lesions are part of target lesions
² Following an initial CR, a PD cannot be assigned if all non-nodal target lesions are still not present and all nodal lesions are <10 mm in size. In this case, the target lesion response is CR.

TABLE E2A
Evaluation of overall cancer response. Acronyms refer to Table E1.

| | | | Objective |
|---|---|---|---|
| Target Lesion | Non-Target Lesion | New Lesion | Status |
| CR | CR | No | CR |
| CR | Non-CR/Non-PD ³ | No | PR |
| CR | Indeterminate or missing | No | PR |
| PR | Non-CR/Non-PD, | No | PR |
| | indeterminate or missing | | |
| SD | Non-CR/Non-PD, | No | SD |
| | indeterminate or missing | | |
| Indeterminate | Non-PD | No | Indeterminate |
| or missing | | | |
| PD | Any | Yes or No | PD |
| Any | PD | Yes or No | PD |
| Any | Any | Yes | PD |

³ Criteria for neither CR nor PD have been met.
All noted regressions should be confirmed ≥4 weeks after criteria for response are first met or at the next tumor assessment.

TABLE E2B
Assignment of time point response using iRECIST. Acronyms refer to Table E1.

| | Timepoint | |
|---|---|---|
| | response | |
| | with no previous | Timepoint response |
| | iUPD in any | with previous |
| | category | iUPD in any category ⁴ |
| Target lesions: iCR; non-target | iCR | iCR |
| lesions: iCR; | | |
| new lesions: no | | |
| Target lesions: iCR; non-target | iPR | iPR |
| lesions: | | |
| non-iCR/non-iUPD; new | | |
| lesions: no | | |
| Target lesions: iPR; non-target | iPR | iPR |
| lesions: | | |
| non-iCR/non-iUPD; new | | |
| lesions: no | | |
| Target lesions: iSD; non-target | iSD | iSD |
| lesions: | | |
| non-iCR/non-iUPD; new | | |
| lesions: no | | |
| Target lesions: iUPD with no | Not applicable | New lesions confirm iCPD if new |
| change, or with a | | lesions were previously identified and |
| decrease from last timepoint; | | they have increased in size (≥5 mm in |
| non-target | | sum of measures for new lesion target |
| lesions: iUPD with no change, | | or any increase for new lesion non- |
| or decrease from last | | target) or number; if no change is seen |
| timepoint; new lesions: yes | | in new lesions (size or number) from |
| | | last timepoint, assignment remains |
| | | iUPD |
| Target lesions: iSD, iPR, iCR; | iUPD | Remains iUPD unless iCPD is |
| non-target lesions: iUPD; new | | confirmed on the basis of a further |
| lesions: no | | increase in the size of non-target |
| | | disease (does not need to meet RECIST |
| | | v1.1 criteria for unequivocal |
| | | progression) |
| Target lesions: iUPD; non- | iUPD | Remains iUPD unless iCPD is |
| target lesions: | | confirmed on the basis of a further |
| non-iCR/non-iUPD, or iCR; | | increase in sum of measures ≥5 mm; |
| new lesions: no | | otherwise, assignment remains iUPD |
| Target lesions: iUPD; non- | iUPD | Remains iUPD unless iCPD is |
| target lesions: iUPD; new | | confirmed based on a further increase |
| lesions: no | | in previously identified target lesion |
| | | iUPD in sum of measures ≥5 mm or |
| | | non-target lesion iUPD (previous |
| | | assessment need not have shown |
| | | unequivocal progression) |
| Target lesions: iUPD; non- | iUPD | Remains iUPD unless iCPD is |
| target lesions: iUPD; new | | confirmed on the basis of a further |
| lesions: yes | | increase in previously identified target |
| | | lesion iUPD sum of measures ≥5 mm, |
| | | previously identified non- target lesion |
| | | iUPD (does not need to be |
| | | unequivocal), or an increase in the size |
| | | or number of new lesions previously |
| | | identified |
| Target lesions: non-iUPD or | iUPD | Remains iUPD unless iCPD is |
| progression; | | confirmed on the basis of an increase in |
| Non-target lesions: non-iUPD | | the size or number of new lesions |
| or progression; new lesions: | | previously identified |
| yes | | |

⁴ Previously identified in assessment immediately before this timepoint. The “i” indicates immune responses assigned using iRECIST.
Abbreviations: iCR = complete response; iPR = partial response; iSD = stable disease; iUPD = unconfirmed progression; non-iCR/non-iPD = criteria for neither CR nor PD have been met; iCPD = confirmed progression; RECIST = Response Evaluation Criteria in Solid Tumors.

TABLE E2C
Overall response in patients with initial measurable
disease and evaluable by CA-125, combining both CA-
125 and RECIST criteria. Acronyms refer to Table E1.

| | Non- | | | Overall | |
|---|---|---|---|---|---|
| Target | target | New | | best | |
| lesion ⁵ | lesion ⁶ | lesion | CA-125 | response | |
| CR | CR | No | Normal | CR | Best RECIST |
| CR | Non CR | No | Not PD | PR | response for |
| | Non PD | | | | this category |
| CR | CR | No | PR not | PR | also requires |
| | | | normal | | it to be confirmed |
| PR | Non PD | No | Not PD | PR | and maintained |
| NE | Non PD | No | PR | PR | for at least |
| PD or New >28 days from | | | PR | PR | 28 days |
| CA-125 PR ⁷ | | | | | |
| SD | Non PD | No | PR | PR | |
| SD | Non PD | No | Not PR | SD | |
| | | | or PD | | |
| PD or New ≤28 days from | | | PR | PD | |
| CA-125 PR ⁷ | | | | | |
| PD | Any | Yes or No | Any | PD | |
| NE | PD | Yes or no | Any | PD | |
| NE | Any | Yes | Any | PD | |
| NE | Any | Yes or no | PD | PD | |

⁵ Target lesions include up to 5 measurable lesions as defined by RECIST.
⁶ Non-target lesions include ascites and peritoneal thickening which are not measurable according to RECIST.
⁷ Patients who have a CA-125 response that occurs more than 28 days from PD according to RECIST are considered a PR according to best response, but PD if the RECIST PD is within 28 days of CA-125 response.
Best RECIST response also requires confirmation and maintenance for at least 28 days.

### Example 2: CD22 Target Engagement

The ability of the administered Compound 1 to exhibit and maintain CD22 target (CD22) engagement was assessed. Using the protocol of Example 1, Compound 1 was administered to patients as a biweekly (Q2W) intravenous infusion over approximately 90 min, at 0.1 mg/kg (1 patient), 0.3 mg/kg (1 patient), 1 mg/kg (7 patients) or 3 mg/kg (8 patients). Peripheral whole blood (PB) was drawn on Cycle 1 before the first dose on Day 1 (C1D1 pre), and after administration at 3 hours (C1D1 3 hrs) then on Days 2 (C1D2), 8 (C1D8), 15 prior to the administration of the second dose (C1D15 pre), as well as blood draws on time points in subsequent cycles.

CD19⁺ B cells from PB were analyzed for surface expression of CD22 by fluorescence-activated cell sorting (FACS) analysis using a Navios flow cytometer (Beckman Coulter) or CytoFLEX LX cytometer (Beckman Coulter). Data analysis was performed using either WinList (Verity Software) or FlowLogic softwares. B cells were identified as CD45⁺CD19⁺ lymphocytes. Molecules of Equivalent Soluble Flurochrome (MESF) values of cell surface CD22 expression were determined using SpheroTech Rainbow Calibration Beads URCP-38-2K (SpheroTech) per manufacturer's recommendation. Data were tabulated using GraphPad Prism software. Data were expressed as mean percent change of CD22 cell surface expression from pre-dose value +/− standard deviation (SD).

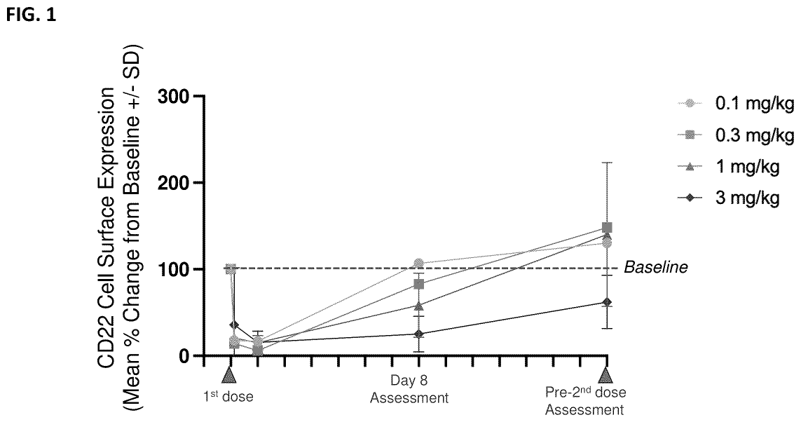

As shown in FIG. 1, CD22 internalization was observed at all dose levels of Compound 1. The higher dose levels exhibited evidence of prolonged target engagement. Surprisingly, while in the lower doses the CD22 cell surface levels returned to baseline in the pre-2^{nd} dose assessment in a fairly linear fashion, at the highest dose level tested in this assay (3 mg/kg), the CD22 cell surface level had not yet returned to base line levels.

### Example 3: B Cell Activation

B cell activation in response to administration of Compound 1 was assessed. The B cells from PB collected from patients in Example 2 were analyzed for activation by measuring CD86 expression by FACS analysis using either a Navios flow cytometer (Beckman Coulter) or a CytoFLEX LX cytometer (Beckman Coulter). Data analysis was performed using either WinList (Verity Software) or FlowLogic softwares. Naïve B cells were identified as CD45⁺CD19⁺CD27⁻ and memory B cells were identified as CD45⁺CD19⁺CD27⁺. CD86 expression was derived by gating on percent of naïve and memory B cells expressing CD86. CD40 values were derived using MESF gated on both naïve and memory B cells. Data were tabulated using GraphPad Prism software and were expressed as fold-change of marker expression from pre-dose.

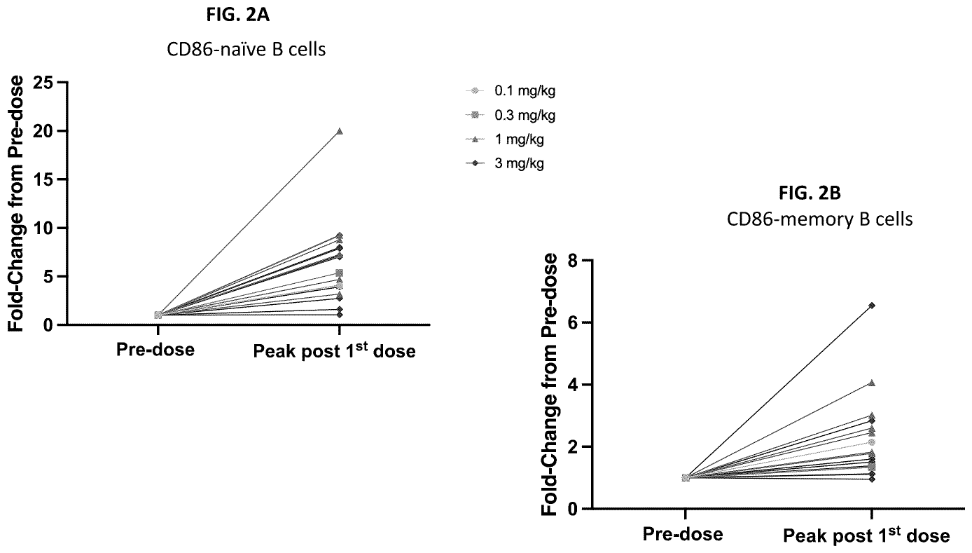

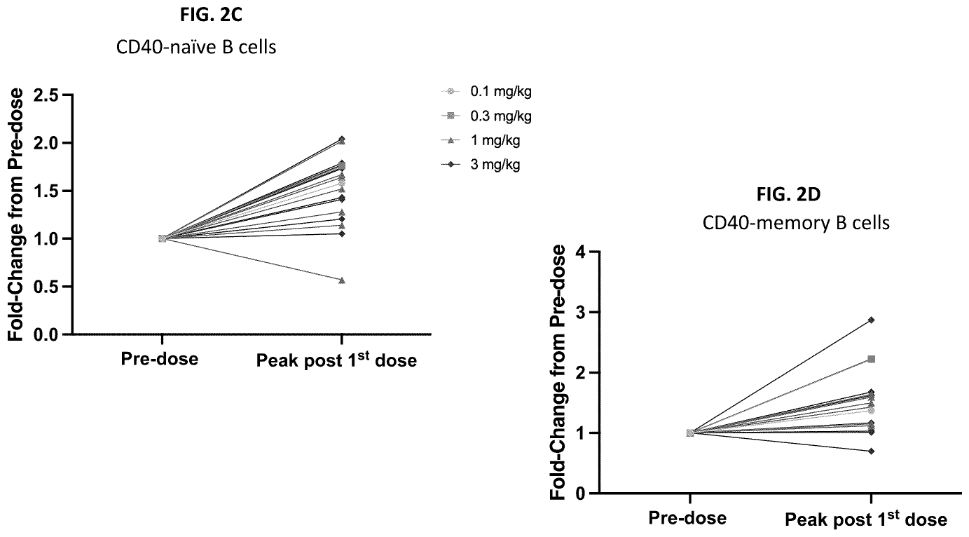

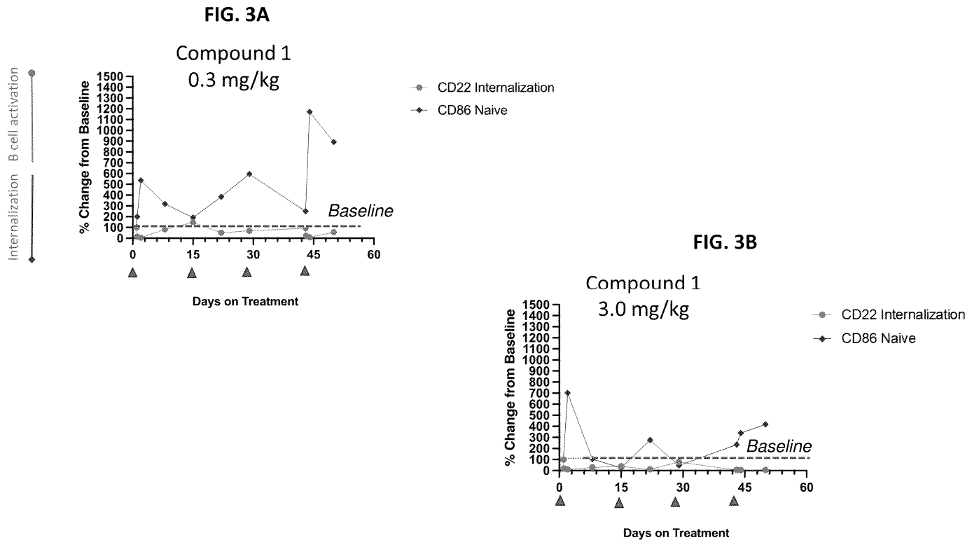

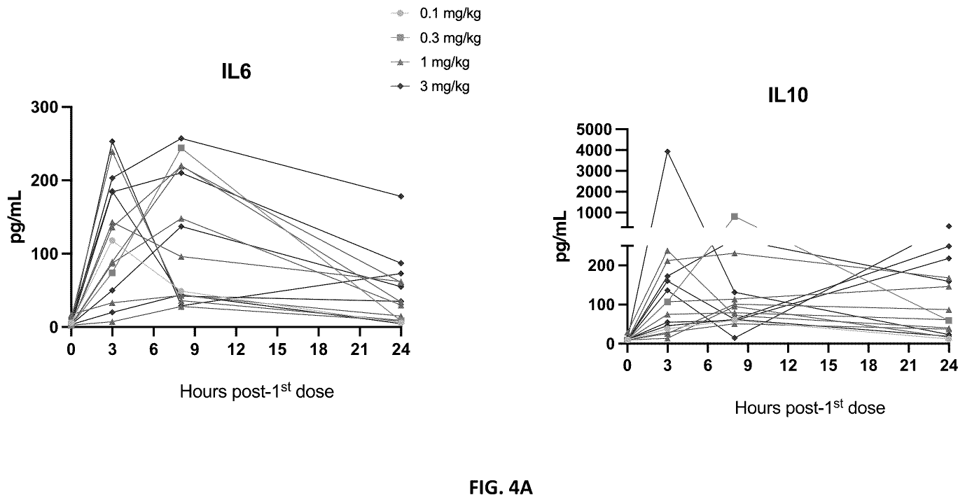

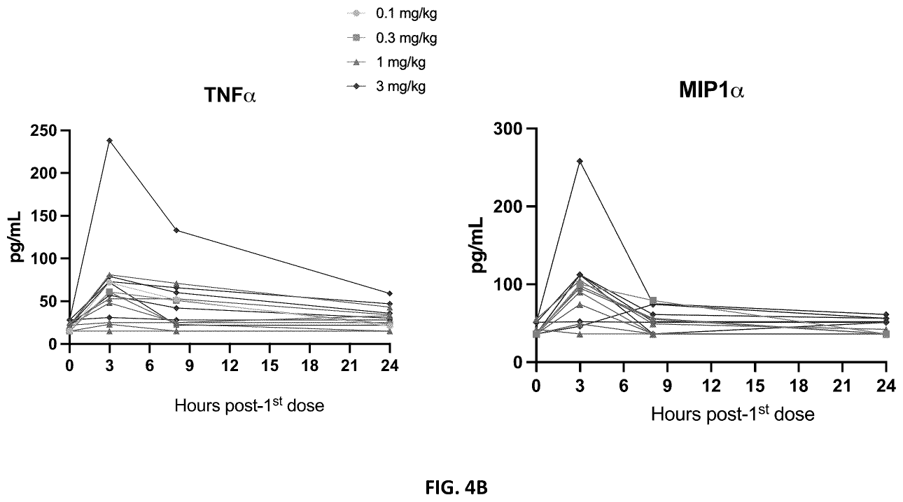

As shown in FIG. 2A and FIG. 2B respectively, after dosing with Compound 1, CD86 expression increased in both naïve and memory B cells, indicating that Compound 1 upregulates expression of the CD86 co-stimulatory marker on naïve and memory B cells in peripheral blood. Expression of CD40 was increased as well after dosing with Compound 1 in both naïve and memory B cells as shown in FIG. 2C and FIG. 2D, respectively. Peak CD40 expression in peripheral blood was assessed post-first dose up to Day 15 pre-second dose. FIGS. 3A and 3B, show the correlation between CD22 internalization and CD86 expression on naïve B cells.

### Example 4: Activation of Peripheral B Cells

Serum cytokines and chemokines were assessed at pre-dose, 3 hr, 8 hr and 24 hr post first dose of Compound 1.

All samples were stored at less than −70° C. until tested on multiplex panels HMPC125 and HMPCORE1 (Rules-Based Medicine). Samples were thawed at room temperature, vortexed, spun at 3700×g for 5 min for clarification and transferred to a master microtiter plate. Using automated pipetting, an aliquot of each sample was added to individual microsphere multiplexes of the selected Multi Analyte Profile (MAP) and blocker. This mixture was thoroughly mixed and incubated at room temperature for 1 hour. Multiplexed cocktails of biotinylated reporter antibodies were added robotically and after thorough mixing, incubated for an additional hour at room temperature. Multiplexes were labelled using an excess of streptavidin-phycoerythrin solution, thoroughly mixed and incubated for 1 hour at room temperature. The volume of each multiplexed reaction was reduced by vacuum filtration and washed 3 times. After the final wash, the volume was increased by addition of buffer for analysis using a Luminex instrument and the resulting data interpreted using proprietary software developed by Rules-Based Medicine. For each multiplex, both calibrators and controls were included on each microtiter plate. Eight-point calibrators to form a standard curve were run in the first and last column of each plate and controls at 3 concentration levels were run in duplicate. Standard curve, control, and sample QC were performed to ensure proper assay performance. Study sample values for each of the analytes were determined using 4 and 5 parameter logistics, weighted and non-weighted curve fitting algorithms included in the data analysis.

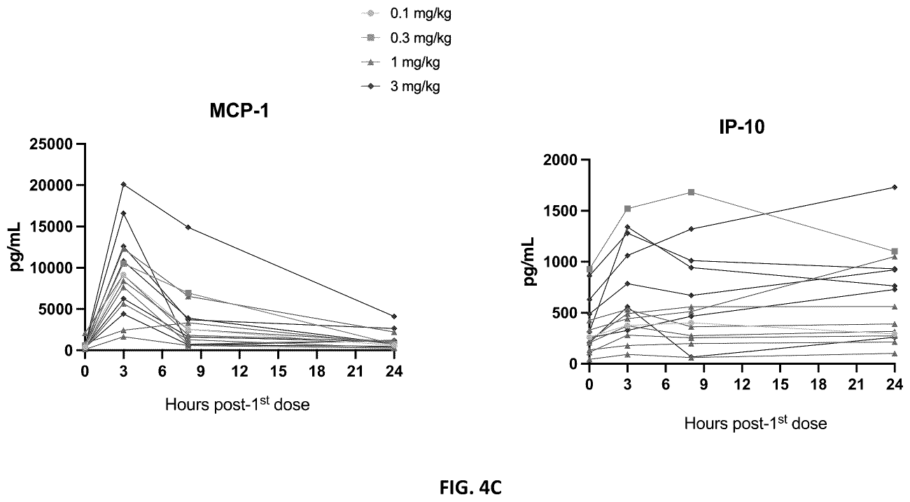

FIGS. 4A-4C show that Compound 1 elicited production of cytokines and chemokines consistent with TLR9-mediated activation of B cells in these patients. Peak levels appear within 24 hours following the first dose, with no evidence of accumulation in serum. Dosing was done at point 0 on each graph. There was not a clear dose-response correlation between the dose level and the cytokine and chemokine levels activated by Compound 1.

### Example 5: Pharmacokinetics

The pharmacokinetics for administration of the compound of Formula I were assessed by measuring the concentration of the conjugate in patients administered 0.1, 0.3, 1.0 and 3 mg/kg from Example 1. A MesoScale Discoveries (MSD) assay was used to detect the conjugate, using a Human CD22 ECD (TNT9, Tallac Therapeutics) ligand binding format and electrochemiluminescence (ECL) technology. The conjugate was detected using a biotin-conjugated anti-sense oligo (primary detection agent) and Sulfo-TAG-labeled Streptavidin. Electrochemiluminescent signal, following the addition of Read Buffer T, was read on an MSD Sector Imager plate reader. The results are provided in Table 1 (AUCₗₐₛₜ, area under the concentration versus time curve from dosing to last measurable concentration; Cₘₐₓ, maximum plasma concentration). Table 1 shows mean values; standard deviation is shown for 1-mg/kg and 3-mg/kg dose groups.

TABLE 1
Pharmacokinetic Parameter Summary at Cycle 1, Day 1

| Dose | | Cₘₐₓ | AUCₗₐₛₜ | |
|---|---|---|---|---|
| mg/kg | n | (μg/mL) | (h*μg/mL) | |
| 0.1 | 1 | 1.74 | 6.28 | |
| 0.3 | 1 | 5.04 | 44.9 | |
| 1 | 7 | 17.9 (5.27) | 205 | (78.4) |
| 3 | 6 | 52.8 (18.2) | 532 | (163) |

As of the date of this filing, one melanoma patient exhibited a partial response at 3 mg/kg given every 2 weeks. This patient is a 56-year-old male with relapsed/refractory metastatic melanoma who had been previously treated with palliative radiation, nivolumab (×16 months) and ipilimumab for (4 doses), with stable disease as the best response. The patient had discontinued nivolumab secondary to disease progression. This patient had a reduction in tumor measurement (based on RECIST criteria) from 56 mm to 39 mm, a 30.6% reduction, as demonstrated by a CT scan taken after week 16 of dosing with Compound 1 as compared to baseline scans taken prior to dosing. Another patient with cholangiocarcinoma dosed with Compound 1 at 0.3 mg/kg given every 2 weeks exhibited durable stable disease >6 months.

### Example 6: Pharmacokinetic-Pharmacodynamic (PK-PD) Analysis

PK-PD analyses for administration were assessed as described in Example 5 for doses of the conjugate (Compound 1) in patients administered 0.1, 0.3, 1, 3, 6 and 12 mg/kg Q2W from Example 1. Average AUCₗₐₛₜ (area under the concentration versus time curve from dosing to last measurable concentration) and average Cₘₐₓ (maximum plasma concentration) were substantially dose proportional. The pharmacokinetics, however, were unexpectedly shorter than modeling performed with non-human primate data.

CD22 internalization assessed at the 6 dosing levels prior to the second round of dosing showed distinct differences in cell surface CD22 expression between the lower and higher three doses. These differences were absent at the assessments performed just prior to the 3^{rd} and 4^{th} dosing cycles. CD22 internalization appeared to reach close to saturation between 3 and 6 mg/kg Q2W. Interestingly, induction of CD86 expression as a B-cell activation marker was observed at all dose levels. Surprisingly, clinically relevant responses of stable disease >6 months were observed in a subject at the 0.3 mg/kg dose, and partial responses were observed in subjects at the 3 and 6 mg/kg doses, suggesting that, with the modality of the conjugate, complete saturation is not necessary for efficacy nor is the modality correlated directly with the pharmacodynamic readouts. These data, along with the safety data from the trial, suggest that doses of the conjugate administered between 0.3-6 mg/kg Q2W exhibited acceptable tolerability and efficacy responses.

NanoString analysis was performed according to the following procedure. Gene expression profiling using the NanoString nCounter platform was performed by Canopy Biosciences—A Bruker Company (St. Louis, MO). RNA was extracted from FFPE curls using the Qiagen RNeasy FFPE Kit. The resulting RNA was quantified on the NanoDrop instrument. For gene expression analysis on the NanoString nCounter system, 100-200 ng of RNA was hybridized to a multiplexed nucleotide probe pool (Human Pan Cancer I0360 Panel) for 16 hours at 65° C. Enriched targets were purified and quantified using the nCounter Dx FLEX Analysis System. Data was analyzed by ROSALIND®, with a HyperScale architecture developed by ROSALIND, Inc. (San Diego, CA). Normalization, fold changes and p-values were calculated using criteria provided by Nanostring. ROSALIND© follows the nCounter® Advanced Analysis protocol of dividing counts within a lane by the geometric mean of the normalizer probes from the same lane. Housekeeping probes to be used for normalization are selected based on the geNorm algorithm as implemented in the NormqPCR R library1. Abundance of various cell populations is calculated on ROSALIND using the Nanostring Cell Type Profiling Module. ROSALIND performs a filtering of Cell Type Profiling results to include results that have scores with a p-Value greater than or equal to 0.05. Fold changes and pValues are calculated using the fast method as described in the nCounter® Advanced Analysis 2.0 User Manual.

NanoString analysis of melanoma tissues from a subject with a partial response to compound 1 (dosed at 3 mg/kg every 2 weeks) showed increased expression of genes related to TLR pathway activation, antigen processing and presentation, cytotoxicity, and TLS induction within the tumor microenvironment of the treated tissue compared to the screening sample. The analysis also indicated higher immune cell abundance in both innate and adaptive responses, alongside a trend of elevated checkpoint pathway gene expression, suggesting potential for combination with checkpoint inhibitors.

### Example 7: Combination Therapy

A clinical study is performed similar to as described in Example 1 with Compound 1 in combination with an anti-PD-1 antibody, pembrolizumab. Both compound 1 and the pembrolizumab are administered to patients every 3 weeks (Q3W) by intravenous infusion to participants with select advanced or metastatic solid tumors at doses shown in Table 2.

TABLE 2
Combination therapy dosing

| Dose level | Compound 1 dose | Pembrolizumab dose |
|---|---|---|
| 0 | 0.3 mg/kg Q3W | 200 mg Q3W |
| 1 | 1.0 mg/kg Q3W | 200 mg Q3W |
| 2 | 3.0 mg/kg Q3W | 200 mg Q3W |
| 3 | 6.0 mg/kg Q3W | 200 mg Q3W |

Safety, tolerability, PK, PD, and preliminary efficacy of the combination are assessed, similar to as described in Examples 1-6. Target patient populations for this study include patients with locally advanced or metastatic disease including: (a) wild type cutaneous melanoma or with cutaneous melanoma with known treatable genetic mutations (e.g., such as BRAF V600, NTRK, KIT); (b) biliary tract cancer (intra-or extrahepatic cholangiocarcinoma or gall bladder cancer) (c) immunogenic solid tumor types: gastric cancer (stomach and gastroesophageal junction), non-small cell lung cancer (NSCLC), renal cell carcinoma (RCC), urothelial cancer, cutaneous squamous cell carcinoma, Merkel cell carcinoma, known microsatellite instable (MSI-H), mismatch repair (MMR) deficient, or tumor mutation burden high (TMB-H, >10 mut/Mb) tumors, and other immunogenic solid tumor types; (d) wild type solid tumors or solid tumors with known treatable genetic mutations (e.g., HER2, BRAF V600, FGFR, IDH1, NTRK, RET, KIT, ROS1, NRAS, ALK, MET, KRASG12C).

What is claimed is:

1. A method of treating cancer in an individual comprising administering to the individual a compound of Formula I at a dose of between 3 mg/kg to 30 mg/kg, wherein the compound of Formula I comprises an anti-CD22 antibody (Ab) and an immunomodulating oligonucleotide (P)
wherein the Ab comprises two antibody light chains, two antibody heavy chains, and two Q-tag peptides (Q); wherein each of the two Q-tag peptides comprises the amino acid sequence RPQGFGPP (SEQ ID NO:2); wherein one Q-tag peptide is linked to the C-terminus of each of the two antibody heavy chains; wherein one of the two Q-tag peptides is linked to the immunomodulating oligonucleotide via an amide bond with the glutamine residue of the Q-tag peptide and a linker (L) as shown in Formula (A): 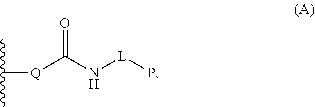
wherein [symbol] indicates the point of attachment of Q to the antibody (Ab);
wherein each heavy chain of the Ab comprises a CDR1 comprising the amino acid sequence of SEQ ID NO:6, a CDR2 comprising the amino acid sequence of SEQ ID NO:7, and a CDR3 comprising the amino acid sequence of SEQ ID NO:8, and each light chain of the Ab comprises a CDR1 comprising the amino acid sequence of SEQ ID NO:9, a CDR2 comprising the amino acid sequence of YTS, and a CDR3 comprising the amino acid sequence of SEQ ID NO: 11;
wherein the linker L is 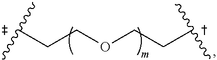
wherein m is 24, and wherein [symbol] † indicates the point of attachment to the oligonucleotide P, and [symbol] ‡ indicates the point of attachment to the glutamine residue of the Q-tag; and
wherein the oligonucleotide P comprises the structure: 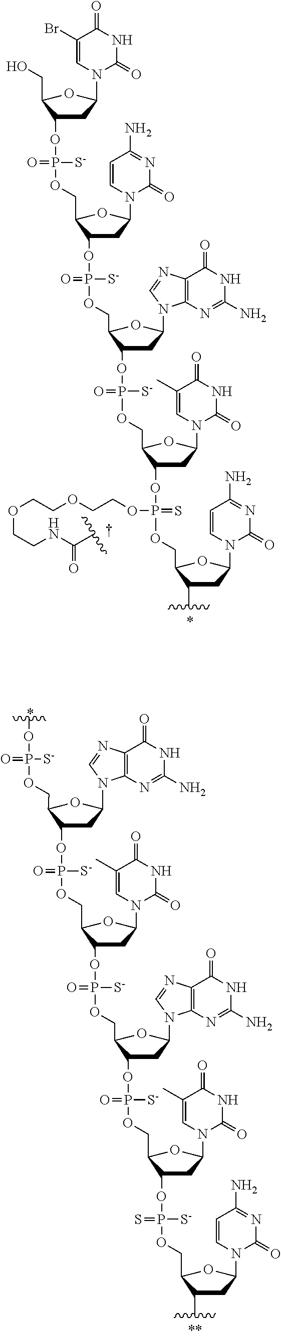 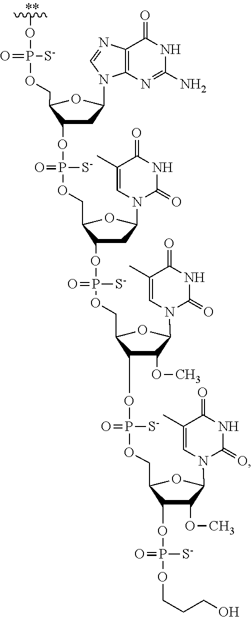
or a pharmaceutically acceptable salt thereof,
wherein [symbol] * and [symbol] ** indicate the points of attachment within the oligonucleotide,
wherein [symbol] † indicates the point of attachment to the linker L.

2. The method of claim 1, wherein the compound of Formula I is administered to the individual at a dose of 3.0, 6.0, 12.0, 18.0, 24.0 or 30.0 mg/kg.

3. The method of claim 1 or claim 2, wherein the compound of Formula I is administered to the individual once every two weeks.

4. The method of claim 1 or claim 2, wherein the compound of Formula I is administered to the individual once every three weeks.

5. The method of claim 1 or claim 2, wherein the compound of Formula I is administered to the individual once every four weeks.

6. The method of any one of claims 1-5, further comprising administering an anti-PD-1 antibody to the individual.

7. The method of claim 6, wherein the anti-PD-1 antibody is pembrolizumab.

8. The method of claim 7, wherein the pembrolizumab is administered to the individual once every three weeks.

9. The method of claim 7 or claim 8, wherein the pembrolizumab is administered to the individual at a dose of 200 mg.

10. The method of claim 1, further comprising administering an anti-PD-1 antibody to the individual, wherein the anti-PD-1 antibody is pembrolizumab, and wherein the pembrolizumab is administered to the individual at a dose of 200 mg once every three weeks and the compound of Formula I is administered to the individual once every three weeks at a dose of 0.3 mg/kg, 1.0 mg/kg, 3.0 mg/kg, or 6.0 mg/kg.

11. The method of any one of claims 1-10, wherein the individual has a solid tumor.

12. The method of claim 11, wherein the solid tumor is inoperable, locally advanced, metastatic, and/or recurrent.

13. The method of any one of claims 1-12, wherein the individual has a cancer selected from the group consisting of melanoma, cutaneous melanoma, breast cancer, ovarian cancer, colorectal cancer, non-small cell lung cancer (NSCLC), renal cell carcinoma (RCC), head and neck squamous cell carcinoma (HNSCC), Merkel cell carcinoma, hepatocellular carcinoma (HCC), cervical squamous cell carcinoma, urothelial carcinoma, endometrial carcinoma, gastro-esophageal adenocarcinoma, and cholangiocarcinoma.

14. The method of any one of claims 1-12, wherein the individual is diagnosed or has cutaneous melanoma.

15. The method of claim 14, wherein tumor cells of the cutaneous melanoma comprise a genetic mutation.

16. The method of claim 15, wherein the genetic mutation is a mutation in one or more of BRAF, NTRK and KIT.

17. The method of claim 14, wherein tumor cells of the cutaneous melanoma do not comprise a genetic mutation.

18. The method of any one of claims 1-12, wherein the individual is diagnosed or has a biliary tract cancer.

19. The method of claim 18, wherein the biliary tract cancer is intra- or extrahepatic cholangiocarcinoma.

20. The method of claim 18, wherein the biliary tract cancer is a gall bladder cancer.

21. The method of any one of claims 1-12, wherein the individual has an immunogenic solid tumor.

22. The method of claim 21, wherein the immunogenic solid tumor is a gastric cancer, a cancer of the stomach and gastroesophageal junction, a non-small cell lung cancer (NSCLC), renal cell carcinoma (RCC), urothelial cancer, cutaneous squamous cell carcinoma, Merkel cell carcinoma, or a tumor with one or more of known microsatellite instable (MSI-H), mismatch repair (MMR) deficient, and tumor mutation burden high.

23. The method of any one of claims 1-12, wherein the individual has a solid tumor or tumor cells with one or more genetic mutations.

24. The method of claim 15, wherein the genetic mutation is a mutation in one or more of HER2, BRAF, FGFR, IDH1, NTRK, RET, KIT, ROS1, NRAS, ALK, MET, and KRAS.

25. The method of any one of claims 1-24, wherein the individual has a solid tumor that has progressed on and/or is intolerant to standard therapy.

26. The method of any one of claims 1-25, wherein the cancer has progressed or the cancer has recurred after prior treatment with an immune-oncology agent.

27. The method of claim 26, wherein the immune-oncology agent is an anti-PD-1 antibody or an anti-CTLA4 antibody.

28. The method of any one of claims 1-27, wherein each heavy chain of the Ab comprises a heavy chain variable region comprising the amino acid sequence of SEQ ID NO:3.

29. The method of any one of claims 1-28, wherein each light chain of the Ab comprises a light chain variable region comprising the amino acid sequence of SEQ ID NO:4.

30. The method of any one of claims 1-29, wherein each heavy chain of the Ab including the Q-tag peptide comprises the amino acid sequence of SEQ ID NO: 14; and wherein each light chain of the Ab comprises the amino acid sequence of SEQ ID NO: 15.

31. The method of any one of claims 1-30, wherein the individual is a human.

32. The method of any one of claims 1-31, wherein the compound of Formula I is administered in a formulation comprising:
(a) the compound of Formula I at a concentration of 30 mg/mL;
(b) 20 mM citrate;
(c) 150 mM L-arginine;
(d) 50 mM NaCl; and
(e) 0.02% polysorbate 80 (w/v); wherein the formulation is at pH 6.5.

33. The method of any one of claims 1-32, wherein the compound of Formula I is administered to the individual intravenously.
